(19) United States
(12) Patent Application Publication
(10) Pub. No.: US 2025/0319201 A1
(43) Pub. Date: Oct. 16, 2025

(54) MUTANT OF ADENO-ASSOCIATED VIRUS AND USE THEREOF

(71) Applicant: PACKGENE BIOTECH CO., LTD., Guangzhou, Guangdong (CN)

(72) Inventors: Huapeng Li, Guangzhou, Guangdong (CN); Ye Bu, Guangzhou, Guangdong (CN); Yujian Zhong, Guangzhou, Guangdong (CN); Zhiyong Dai, Guangzhou, Guangdong (CN); Youwei Zhang, Guangzhou, Guangdong (CN); Yue Pan, Guangzhou, Guangdong (CN); Huan Chen, Guangzhou, Guangdong (CN)

(21) Appl. No.: 19/253,966

(22) Filed: Jul. 10, 2025

Related U.S. Application Data
(63) Continuation of application No. PCT/CN2023/072508, filed on Jan. 17, 2023.

(30) Foreign Application Priority Data
Dec. 30, 2022 (CN) 202211731065.4

(51) Int. Cl. A61K 48/00; C07K 14/005; C12N 15/86

(52) U.S. Cl. CPC A61K 48/0041; C07K 14/005; C12N 15/86; C12N 2750/14122; C12N 2750/14143; C12N 2750/14151

(57) ABSTRACT

The present disclosure provides a mutant of an adeno-associated virus 2 (AAV2) capsid protein, including an amino acid sequence KTINGSGQNQQTLK (SEQ ID NO: 2) or an amino acid sequence having 1, 2, 3, or 4 amino acid changes when compared to SEQ ID NO: 2 in variable region IV; and an amino acid sequence TTVTQ (SEQ ID NO: 3) or an amino acid sequence having 1 or 2 amino acid changes when compared to SEQ ID NO: 3 in variable region V. The mutants of an adeno-associated virus provided herein have low liver tropism and low hepatotoxicity.

### CROSS-REFERENCE TO RELATED APPLICATIONS

The present application is a Continuation application of PCT application No. PCT/CN2023/072508 filed on Jan. 17, 2023, which claims the benefit of Chinese Patent Application No. 202211731065.4 filed on Dec. 30, 2022. The contents of the above-identified applications are hereby incorporated by reference.

### REFERENCE TO SEQUENCE LISTING

This application includes a Sequence Listing filed electronically as an XML file named “U.S. Pat. No. 2,401,291H-PCT_SL.xml”, created on Apr. 11, 2025, with a size of 35,672 bytes. The Sequence Listing is incorporated herein by reference.

### TECHNICAL FIELD

The present application relates to virions and applications thereof in the field of biology, and in particular to a mutant of an adeno-associated virus (AAV) having low liver tropism and high specificity, and applications thereof.

### BACKGROUND

Adeno-associated virus (AAV) is a small non-enveloped virus encapsulating a linear single-stranded DNA genome. It belongs to the genus Dependovirus of the family Parvoviridae and requires a helper virus (usually an adenovirus) to participate in replication. The AAV genome is a single-stranded DNA fragment contained in a capsid of a non-enveloped virus and can be divided into three functional regions: two open reading frames (Rep gene, Cap gene) and the inverted terminal repeat (ITR) sequence. Recombinant adeno-associated viral (rAAV) vectors are derived from a wild-type adeno-associated virus which is non-pathogenic. Because rAAV has the advantages of a wide host range, non-pathogenicity, low immunogenicity, long-term stable expression of exogenous genes, good diffusion performance, and stable physical properties, it is widely used as a gene transfer vector in gene therapy and vaccine research. In medical research, rAAV is used in gene therapy research for a variety of diseases (including in vivo and in vitro experiments), such as gene function research, disease model construction, and preparation of gene knockout mice.

Currently, a variety of AAV vectors have been widely used in clinical trials, among which the most frequently used is AAV2, such as the marketed drug Luxturna. Other newer and more potent capsids, such as AAV8, AAV9, and AAVrh.10, are being used in a growing number of trials. Although there are many serotypes available for selection, each serotype has certain defects, especially adverse reactions or death caused by hepatotoxicity, which are key points. For example, Novartis reported that pediatric patients died of acute liver failure after receiving Zolgensma gene therapy for spinal muscular atrophy (SMA). Homology Medicines announced that the FDA had suspended the clinical trial of HMI-102, a therapy for adult patients with phenylketonuria (PKU), due to abnormal liver function test results of a subject. Similarly, Astellas' gene therapy AT132, which was used to treat X-linked myotubular myopathy, employed an AAV8 vector to deliver the myotubularin gene to the skeletal muscle, thereby increasing the expression of myotubularin in the tissue. The trial was suspended several times, one of the reasons being severe side effects such as liver toxicity that could not be resolved.

AAV2 is one of the earliest serotypes discovered and studied by humans. In the past few decades of research, AAV2 is also the serotype that has been studied most clearly. Compared with AAV2, AAV9 is more efficient in vivo and can effectively infect a variety of tissues. However, one of the shortcomings of AAV9 is its poor specificity. It can target multiple tissues and organs at the same time, especially the liver. For example, the above-mentioned drug Zolgensma utilizes AAV9 as its vector.

In summary, although AAV is one of the safest gene therapy vectors and has been widely used in the field of gene therapy, it is hampered by issues of specificity, especially liver toxicity. Many clinical medications based on AAV have been forced to be interrupted or even led to the death of the subjects. Therefore, developing an AAV having low liver tropism and good specificity, or incorporating specific targeting peptides based on the AAV “backbone” to achieve a more specific, low-toxic, and high-efficiency goal, will have huge clinical value and commercial application scenarios.

### SUMMARY

In one aspect, the present application provides a mutant of an adeno-associated virus 2 (AAV2) capsid protein, including an amino acid sequence KTINGSGQNQQTLK (SEQ ID NO: 2) or an amino acid sequence having 1, 2, 3, or 4 amino acid changes when compared to SEQ ID NO: 2 in variable region IV; and an amino acid sequence TTVTQ (SEQ ID NO: 3) or an amino acid sequence having 1 or 2 amino acid changes when compared to SEQ ID NO: 3 in variable region V.

In some embodiments, 12-16 consecutive amino acids in the variable region IV of a wild-type AAV2 capsid protein are replaced by the amino acid sequence KTINGSGQNQQTLK (SEQ ID NO: 2) or the amino acid sequence having 1, 2, 3, or 4 amino acid changes when compared to SEQ ID NO: 2, and 4-6 consecutive amino acids in the variable region V of the wild-type AAV2 capsid protein are replaced by the amino acid sequence TTVTQ (SEQ ID NO: 3) or the amino acid sequence having 1 or 2 amino acid changes when compared to SEQ ID NO: 3.

In some embodiments of the mutant of the AAV2 capsid protein, amino acids at positions 447-461 of the wild-type AAV2 capsid protein are replaced by the amino acid sequence KTINGSGQNQQTLK (SEQ ID NO: 2) or the amino acid sequence having 1, 2, 3, or 4 amino acid changes when compared to SEQ ID NO: 2, and amino acids at positions 490-494 of the wild-type AAV2 capsid protein are replaced by the amino acid sequence TTVTQ (SEQ ID NO: 3) or the amino acid sequence having 1 or 2 amino acid changes when compared to SEQ ID NO: 3, where the positions of the amino acids correspond to positions in an amino acid sequence of the wild-type VP1 protein set forth in SEQ ID NO: 1.

In some embodiments of the mutant of the AAV2 capsid protein, an amino acid sequence RTNTPSGTTTQSRLQ (SEQ ID NO: 4) in the variable region IV of the wild-type AAV2 capsid protein is replaced by the amino acid sequence KTINGSGQNQQTLK (SEQ ID NO: 2) or the amino acid sequence having 1, 2, 3, or 4 amino acid changes when compared to SEQ ID NO: 2, and an amino acid sequence KTSAD (SEQ ID NO: 5) in the variable region V of the wild-type AAV2 capsid protein is replaced by the amino acid sequence TTVTQ (SEQ ID NO: 3) or the amino acid sequence having 1 or 2 amino acid changes when compared to SEQ ID NO: 3.

In some embodiments of the mutant of the AAV2 capsid protein, the amino acid at position 585 is a non-basic amino acid, where the position of the amino acid corresponds to a position in the amino acid sequence of the wild-type VP1 protein set forth in SEQ ID NO: 1.

In some embodiments of the mutant of the AAV2 capsid protein, arginine (R) at position 585 of the wild-type AAV2 capsid protein is mutated to alanine (A), where the position of the arginine or alanine corresponds to a position in the amino acid sequence of the wild-type VP1 protein set forth in SEQ ID NO: 1.

In some embodiments of the mutant of the AAV2 capsid protein, amino acids at positions 585-587 of the wild-type AAV2 capsid protein are deleted, and wherein the positions of the amino acids correspond to positions in the amino acid sequence of the wild-type VP1 protein set forth in SEQ ID NO: 1.

In some embodiments, the mutant of the AAV2 capsid protein is a mutant of capsid protein VP1, VP2 and/or VP3.

In some embodiments, the mutant of the AAV2 capsid protein includes an amino acid sequence set forth in any one of SEQ ID NOs: 6-8, or an amino acid sequence having at least 80%, at least 90%, at least 95%, or at least 98% sequence identity to the amino acid sequence set forth in any one of SEQ ID NOs: 6-8.

In another aspect, the present application provides an isolated nucleic acid molecule encoding the mutant of the AAV2 capsid protein.

In some embodiments, the isolated nucleic acid molecule includes an nucleotide sequence set forth in any one of SEQ ID NOs: 10-12.

In another aspect, the present application provides an expression vector including the isolated nucleic acid molecule above.

In another aspect, the present application provides a host cell including the isolated nucleic acid molecule or the expression vector above.

In another aspect, the present application provides a host cell, which expresses the mutant of the AAV2 capsid protein above.

In another aspect, the present application provides an adeno-associated virus (AAV) including the mutant of the AAV2 capsid protein.

In another aspect, the present application provides a method for preparing a recombinant adeno-associated virus (rAAV), including introducing at least the following components into a host cell:
1) the isolated nucleic acid molecule above or an expression vector described herein; and
2) a GOI (gene of interest) plasmid including a target gene.

In some embodiments, the expression product of the target gene is protein or RNA.

In another aspect, the present application provides a rAAV prepared by the method above.

In some embodiments, the rAAV has lower targeting to liver than a wild-type AAV2 or wild-type AAV9.

In some embodiments, the rAAV has higher targeting to muscle, heart, brain, spinal cord, lung, kidney, or eye than the wild-type AAV2 or AAV9.

In another aspect, the present application provides a pharmaceutical composition including the rAAV above and a pharmaceutically acceptable carrier.

In another aspect, the present application provides the use of the isolated nucleic acid molecule, expression vector, or rAAV above in the preparation of drugs.

In another aspect, the present application provides a pharmaceutical composition including the isolated nucleic acid molecule or the expression vector above and a pharmaceutically acceptable carrier.

In some embodiments, the present application provides a method for treating a muscle, heart, brain, spinal cord, lung, kidney, or eye-related disease, including administering a therapeutically effective amount of the pharmaceutical composition to a patient suffering from the muscle, heart, brain, spinal cord, lung, kidney, or eye-related disease, wherein the pharmaceutical composition comprises a heterologous polynucleotide encoding a heterologous gene product.

The mutant of an adeno-associated virus provided in the present application has low liver tropism, low liver toxicity, and better specificity. The recombinant adeno-associated virus vector constructed using the mutant of the AAV capsid protein provided in the present application has higher specificity, better safety, and a wide range of applications.

### BRIEF DESCRIPTION OF THE DRAWINGS

FIGS. 1A and 1B show analysis results of the targeting of different serotypes to livers of C57 mice (4 weeks after virus injection), where FIG. 1A shows the relative expression levels of mRNA in the livers; and FIG. 1B shows the relative expression levels of proteins in the livers.

FIGS. 2A, 2B, and 2C show analysis results of the targeting of different serotypes to quadriceps (quadriceps femoris) of C57 mice (4 weeks after virus injection), where FIG. 2A shows the relative expression levels of mRNA in the quadriceps; FIG. 2B shows a ratio of the mRNA level in the quadriceps to the mRNA level in the liver (i.e., quadriceps/liver ratio (mRNA expression level)); and FIG. 2C shows the relative expression levels of proteins in the quadriceps.

FIGS. 3A, 3B, and 3C show analysis results of the targeting of different serotypes to hearts of C57 mice (4 weeks after virus injection), where FIG. 3A shows the relative expression levels of mRNA in the hearts; FIG. 3B shows a ratio of the mRNA level in the heart to the mRNA level in the liver (i.e., heart/liver ratio (mRNA expression level)); and FIG. 3C shows the relative expression levels of proteins in the hearts.

FIGS. 4A and 4B show analysis results of the targeting of different serotypes to abdominal muscles of C57 mice (4 weeks after virus injection), where FIG. 4A shows the relative expression levels of mRNA in the abdominal muscles; and FIG. 4B shows a ratio of the mRNA level in the abdominal muscle to the mRNA level in the liver (i.e., abdominal muscle/liver ratio (mRNA expression level)).

FIGS. 5A and 5B show analysis results of the targeting of different serotypes to brains of C57 mice (4 weeks after virus injection), where FIG. 5A shows the relative expression levels of mRNA in the brains; and FIG. 5B shows a ratio of the mRNA level in the brain to the mRNA level in the liver (i.e., brain/liver ratio (mRNA expression level)).

FIGS. 6A and 6B show analysis results of the targeting of different serotypes to spinal cords of C57 mice (4 weeks after virus injection), where FIG. 6A shows the relative expression levels of mRNA in the spinal cords; and FIG. 6B shows a ratio of the mRNA level in the spinal cord to the mRNA level in the liver (i.e., spinal cord/liver ratio (mRNA expression level)).

FIGS. 7A, 7B, and 7C show the analysis results of the targeting of different serotypes to lungs, kidneys, and eyes of C57 mice (4 weeks after virus injection), where FIG. 7A shows the relative expression levels of mRNA in the lungs; FIG. 7B shows the relative expression levels of mRNA in the kidneys; and FIG. 7C shows the relative expression levels of mRNA in the eyes.

### DETAILED DESCRIPTION

Unless otherwise indicated, all technical and scientific terms used herein have the meanings commonly understood by those of ordinary skill in the art.

The term “or” refers to a single element of the enumerated optional elements, unless the context clearly indicates otherwise. The term “and/or” refers to any one, any two, any three, any more or all of the optional elements listed.

The terms “comprise” or “include” mean the inclusion of said elements, integers, or steps, but not the exclusion of any other elements, integers, or steps. When the term “comprise” or “include” is used herein, unless otherwise specified, the term also encompasses cases consisting of the recited elements, integers, or steps. For example, reference to a polypeptide “comprising” a particular sequence is intended to encompass polypeptides consisting of that particular sequence.

“Adeno-associated virus (AAV)” is a non-enveloped icosahedral capsid virus of the Parvoviridae family, including a viral genome of single-stranded DNA. The Parvoviridae family includes the genus Dependovirus, which includes AAV, and is dependent on the presence of a helper virus, such as adenovirus, for its replication. Due to its relatively simple structure, ability to infect a variety of cells (including quiescent and dividing cells) without integrating into the host genome, and its relatively mild immunogenicity, AAV has been demonstrated to be useful as a biological tool and for expressing a target gene in vitro or in vivo. Also considered herein are AAV-based expression vectors, including recombinant AAV (rAAV) carrying the target gene and used for therapeutic purposes.

The wild-type AAV viral genome is a linear, single-stranded DNA (ssDNA) molecule that is approximately 5,000 nucleotides (nt) in length. The AAV viral genome usually includes two inverted terminal repeat (ITR) sequences, which cap the viral genome at the 5′ and 3′ ends, respectively, and provide a replication origin for the viral genome. These ITRs have a characteristic T-shaped hairpin structure and have multiple functions, including but not limited to serving as an origin of DNA replication by acting as a primer for the endogenous DNA polymerase complex of the host cell during viral replication.

The wild-type AAV genome also includes the Rep gene and the Cap gene, which encode four non-structural Rep proteins (Rep78, Rep68, Rep52, and Rep40) and three capsid proteins or structural proteins (VP1, VP2, and VP3), respectively. The Rep protein is involved in viral replication and packaging, while capsid proteins are assembled to form the AAV protein shell or AAV capsid. Alternative splicing and alternate start codons and promoters result in the production of four different Rep proteins from a single open reading frame in the Rep gene and three capsid proteins from a single open reading frame in the Cap gene.

In the context of AAV, the term “viral capsid protein” or “capsid protein” is used to refer to the protein of AAV that is capable of self-assembling to produce AAV particles, and is also known as the coat protein or VP protein. The VP protein includes three subunits, VP1, VP2, and VP3, and thus changes in the mutant of the VP protein relative to the wild-type VP protein can be reflected in amino acid sequence changes in the VP1, VP2, and VP3 subunits. Accordingly, as used herein, “mutant of the (AAV) capsid protein” includes a mutant of a VP protein, but also includes a mutant of VP1, VP2, and/or VP3 subunit. Due to the consistency of amino acid sequences between VP1, VP2, and VP3 subunits expressed from the same Cap gene, when changes are made to the coding sequence in the Cap gene, for example, to the coding sequence of the VP3 subunit, the amino acid sequences of the expressed VP1 and VP2 subunits at the same time are altered.

The term “serotype” in the context of AAV is used to refer to the serological distinction of the capsid protein of AAV from other AAV serotypes. Serological uniqueness is determined based on the reactivity of an antibody with one AAV and the lack of cross-reactivity with other or another AAV. Such cross-reactivity differences are usually due to differences in capsid protein sequences (or subunit sequences thereof)/antigenic determinants (e.g., due to differences in VP1, VP2, and/or VP3 sequences of serotype AAV2). A variety of AAV serotypes have been identified, including but not limited to AAV1, AAV2, AAV3, AAV4, AAV5, AAV6, AAV7, AAV8, AAV9, AAV10, AAV11, and AAV12, as well as their mutants.

When referring to the capsid protein or its subunits of AAV, the term “variable region” refers to the region where the amino acid sequence varies relatively significantly between different serotypes. Usually, relatively conserved regions are identified by aligning amino acid sequences of capsid proteins from multiple AAV serotypes, and the sequences between the relatively conserved regions are the variable region sequences. The variable region may be associated with the binding of AAV to cell surface receptors. For the AAV2 serotype, multiple variable regions may be included, such as variable region I to variable region IX (or also referred to as loop I to loop IX). In a preferred embodiment, the positions of variable region IV and variable region V in the AAV2 capsid protein are determined according to the region determination method established by Bennett et al. (Bennett A, Keravala A, Makal V, et al. Structure comparison of the chimeric AAV2.7m8 vector with parental AAV2. J Struct Biol. 2020; 209 (2): 107433).

“Recombinant AAV vector” means an AAV genome derived by removing a portion of wild-type genes (e.g., the Rep gene and Cap gene) from the AAV genome using molecular biology methods and replacing them with heterologous nucleic acid sequences (e.g., coding sequences of proteins or RNAs used for therapeutic purposes). Typically, for a recombinant AAV vector, one or two inverted terminal repeat (ITR) sequences of the AAV genome are retained therein. Most often, recombinant AAV vectors are replication-deficient, lacking sequences encoding functional Rep and Cap proteins in their viral genomes. These replication-deficient AAV particles may lack most of the parental coding sequences and carry essentially only one or two AAV ITR sequences and target nucleic acids for delivery to a cell, tissue, organ, or organism. An AAV including a recombinant AAV vector is referred to herein as a recombinant AAV (rAAV).

The term “GOI (gene of interest) plasmid” as used herein refers to a plasmid that is introduced into a host cell together with a helper plasmid and/or a helper virus when preparing a recombinant AAV, and the GOI plasmid carries a target gene and ITR sequences located on both sides of the target gene. In order to facilitate the expression of the prepared recombinant AAV particles in vivo or in vitro, the target gene (a coding sequence for a protein or RNA) is usually operably linked to a regulatory sequence related to expression, such as a promoter and a polyadenylation (polyA) tailing signal. The term “operably linked” refers to a linkage between polynucleotide elements that places them in a functional relationship. When a nucleic acid or polynucleotide sequence is placed into a functional relationship with another nucleic acid sequence, they are “operably linked”. For example, a transcriptional regulatory sequence (such as a promoter, an enhancer, or other expression control elements known in the art) is operably linked to a coding sequence if it affects the transcription of the coding sequence.

“Amino acid changes” herein include amino acid substitutions, deletions or insertions. The number of amino acid changes in the mutant sequence relative to the parent sequence can be calculated as the sum of the number of amino acid substitutions, the number of amino acid deletions, and the number of amino acid insertions.

As used herein, the terms “nucleic acid molecule”, “nucleic acid”, and “polynucleotide” are used interchangeably to refer to a polymer of nucleotides. Such nucleotide polymers may contain natural and/or non-natural nucleotides and include, but are not limited to, DNA, RNA, and peptide nucleic acids (PNA). The term “nucleic acid sequence” refers to a linear sequence of nucleotides contained in a nucleic acid molecule or polynucleotide. The term “isolated nucleic acid molecule” refers to a nucleic acid molecule that is separated from its natural environment (such as the intracellular environment) and is essentially free of one or more substances that are normally associated with it in nature, such as proteins, nucleic acids, lipids, carbohydrates, cell membranes, etc., or is an artificially prepared (such as artificially synthesized) nucleic acid molecule.

The term “expression vector” refers to a nucleic acid molecule including various expression elements for expressing a target protein or target RNA in a host cell. For expression vectors used to express target proteins in eukaryotic cells, these expression elements generally include promoters, enhancers, polyadenylation signal sequences, and the like. In order to facilitate amplification in E. coli, the expression vector usually also includes an E. coli replicon sequence. In addition, the expression vector may also include an antibiotic resistance gene or a selection marker gene for screening (e.g., ampicillin resistance gene (AmpR), thymidine kinase gene (TK), kanamycin resistance gene (KanR), neomycin resistance gene (NeoR), etc.) and a multiple cloning site (MCS) for inserting the target gene.

The term “host cell” refers to a cell in which an expression vector can be maintained and/or replicated, including a prokaryotic cell and a eukaryotic cell, such as a bacterium (such as E. coli), a fungus (yeast), an insect cell (such as SF9) and a mammalian cell (such as HEK-293T).

When referring to a pharmaceutical composition, the term “pharmaceutically acceptable carrier” refers to a solid or liquid diluent, filler, antioxidant, stabilizer, or other substances that can be safely administered. These substances are suitable for administration to humans and/or animals without undue adverse side effects while being suitable for maintaining the activity of the drug or active agent located therein. According to the route of administration, various different carriers well known in the art can be administered, including, but not limited to, sugars, starch, cellulose and its derivatives, maltose, gelatin, talc, calcium sulfate, vegetable oils, synthetic oils, polyols, alginic acid, phosphate buffer, emulsifiers, isotonic saline, and/or pyrogen-free water, etc.

The “targeting” or “tropism” of AAV or rAAV refers to the phenomenon that when AAV or rAAV is introduced into the body, it relatively accumulates in a specific tissue or organ. For example, targeting or tropism can be manifested as a higher concentration in tissue A than in tissue B. This targeting or tropism can be reflected by detecting the content or concentration of its genome in different tissues or organs.

When referring to amino acid or nucleotide sequences, the term “sequence identity” (also known as “sequence consistency”) refers to a measure of the degree of identity between two amino acid or nucleotide sequences (e.g., a query sequence and a reference sequence), generally expressed as a percentage. Typically, before calculating the percent identity between two amino acid or nucleotide sequences, the sequence alignment is first conducted and gaps (if any) are introduced. If the amino acid residues or bases in the two sequences are the same at a certain alignment position, the two sequences are considered to be consistent or matched at that position; if the amino acid residues or bases in the two sequences are different, they are considered to be inconsistent or mismatched at that position. In some algorithms, the number of matching positions is divided by the total number of positions in the alignment window to obtain sequence identity. In other algorithms, the number of gaps and/or the length of the gaps are also taken into account. Commonly used sequence alignment algorithms or software include DANMAN, CLUSTALW, MAFFT, BLAST, MUSCLE, etc. For the purpose of this application, the publicly available alignment software BLAST (available from https://www.ncbi.nlm.nih.gov/) can be used to obtain the best sequence alignment and calculate the sequence identity between two amino acid or nucleotide sequences using the default settings.

The present application is based, at least in part, on the finding that substitution of a portion of the sequence of the AAV2 capsid protein (hereinafter referred to as the “substituted sequence”) results in a mutant of the capsid protein with altered targeting to an organ, and in particular, reduced liver tropism. The sequence used for substitution (hereinafter referred to as the “substitution sequence”, i.e., a “substituted sequence” is replaced with a “substitution sequence”) may be derived from capsid proteins of other serotypes. In some embodiments, the substitution sequence used is from the AAV9 capsid protein. In some embodiments, the substitution sequence is KTINGSGQNQQTLK (SEQ ID NO: 2). In other embodiments, the substitution sequence is an amino acid sequence having 1, 2, 3, or 4 amino acid changes when compared to SEQ ID NO: 2. In some embodiments, the substitution sequence is TTVTQ (SEQ ID NO: 3). In other embodiments, the substitution sequence is an amino acid sequence having 1 or 2 amino acid changes when compared to SEQ ID NO: 3. In some embodiments, sequence substitutions are performed at two or more positions of the AAV2 capsid protein. In some embodiments, the substituted sequence is located in the variable region IV. In other embodiments, the substituted sequence is located in the variable region V. In some embodiments, the substituted sequence is located in the variable region IV and the variable region V. Preferably, the sequence SEQ ID NO: 4 of the capsid protein of wild-type AAV2 may be replaced with a substitution sequence SEQ ID NO: 2 or an amino acid sequence having 1, 2, 3, or 4 amino acid changes when compared to SEQ ID NO: 2. Preferably, the sequence SEQ ID NO: 5 of the capsid protein of wild-type AAV2 can be replaced with the substitution sequence SEQ ID NO: 3 or the amino acid sequence having 1 or 2 amino acid changes when compared to SEQ ID NO: 3. More preferably, the sequence SEQ ID NO: 4 of the capsid protein of wild-type AAV2 can be replaced with the substitution sequence SEQ ID NO: 2 or the amino acid sequence having 1, 2, 3, or 4 amino acid changes when compared to SEQ ID NO: 2, and the sequence SEQ ID NO: 5 of the capsid protein of wild-type AAV2 can be replaced with the substitution sequence SEQ ID NO: 3 or the amino acid sequence having 1 or 2 amino acid changes when compared to SEQ ID NO: 3. Most preferably, the sequence SEQ ID NO: 4 of the capsid protein of wild-type AAV2 can be replaced with the substitution sequence SEQ ID NO: 2, and the sequence SEQ ID NO: 5 of the capsid protein of wild-type AAV2 can be replaced with the substitution sequence SEQ ID NO: 3.

The present application also relates to further mutations based on the above mutants to obtain mutants of the capsid protein having further optimized targeting. In some embodiments, the mutant comprises a mutant amino acid at position 585. Specifically, the mutation R585A may be included, or a 3-amino acid-deletion starting from position 585, such as a RGN deletion, may be included.

When referring to specific amino acid positions herein, they are all positions corresponding to the amino acid sequence of the wild-type VP1 protein set forth in SEQ ID NO: 1.

Accordingly, the present application provides three recombinant adeno-associated virus (rAAV) virions having mutant capsid proteins, and demonstrates targeting characteristics of these virions to different organs. Among them, mutant 1 has liver tropism far lower than its parental AAV2 and AAV9, and is the first publicly disclosed method of using this sequence feature to modify and reduce liver tropism. Mutants 2 and 3 have superior muscle/liver ratios (i.e., ratios of an mRNA and/or protein level in the muscle to an mRNA and/or protein level in the liver). In particular, regarding mutant 2, although its main modified sequence is derived from AAV2, mutant 2 has the ability to target muscles and hearts similar to AAV9 and has better tropism for the spinal cord. These mutants will generate huge social value and economic benefits based on their respective targeting characteristics or the properties of new mutants derived from their common backbone.

Specifically, the present application provides a mutant of an AAV capsid protein having low targeting to liver, wherein:
(a) The amino acid sequence of the mutant is a sequence set forth in any one of SEQ ID NOs: 6-8; or
(b) The mutant is a protein comprising substitution, deletion, or addition of one or more amino acids in the amino acid sequence set forth in any one of SEQ ID NOs: 6-8 and having the activity of the mutant defined in (a).

Also provided herein is a recombinant adeno-associated virus virion, including:
(a) the mutant of the AAV capsid protein above; and
(b) a heterologous polynucleotide encoding a heterologous gene product.

In some embodiments, the gene product is a polypeptide.

In other embodiments, the gene product is selected from the group consisting of an interfering RNA, an aptamer, an endonuclease, and a guide RNA.

Also provided herein is the use of the above-mentioned recombinant adeno-associated virus virion in the preparation of a medicament for delivering a gene product to a cells or a tissue of a subject.

The present application will be further described below in conjunction with specific embodiments, and the advantages and features of the present application will become clearer as the description proceeds. These embodiments are merely exemplary and do not limit the scope of the present application in any way. Those skilled in the art should understand that the details and forms of the technical solutions of the present application may be modified or replaced without departing from the spirit and scope of the present application, but these modifications and replacements are within the scope of the present application.

### Example 1: Design, Construction, and Virus Production of AAV Mutants

### 1) Design of AAV Mutants:

The inventors analyzed the three-dimensional structures of AAV2 (PDB: 6IH9) and AAV9 (PDB: 3UX1) and literature data, and replaced the key sites related to targeting binding with an AAV2 receptor, such as IV loop (AAV2: R447-Q461 replaced by AAV9: K449-K462) and V loop (AAV2: K490-D494 replaced by AAV9: T491-Q495), to obtain mutant 1 of the target serotype (SEQ ID NO: 6). Since R585 of AAV2 is the key amino acid site for binding to heparan sulfate proteoglycan (HSPG), and HSPG is one of the main receptors mediating the liver tropism of AAV2, the inventors further mutated this to form mutant 2 (AAV2: R585 mutated to A585) (SEQ ID NO: 7) and mutant 3 (AAV2: deletion mutation of R585GN) (SEQ ID NO: 8), and further conducted relevant in vivo activity tests in animals.

### 2) Construction of Mutant Serotype Vector and Plasmid Extraction:

The Rep-CAP plasmid was double-digested with Smil and BshTI, subjected to gel electrophoresis, and a fragment band of about 5000 bp was cut out for gel recovery to obtain a digested backbone fragment.

According to the Cap sequence of mutant 1, the following primers were designed, and the specific steps included: The Rep-CAP plasmid of AAV2 was used as a template and the primers Cap-f+YJ69-R were used for amplification; then the amplified product was recovered by gel extraction to obtain the target product 246-1. The Rep-CAP plasmid of AAV2 was used as a template and primers YJ69-F+YJ72-R were used for amplification; then the amplified product was recovered by gel extraction to obtain the target product 246-2. The Rep-CAP plasmid of AAV2 was used as a template and YJ72-F+cap-r primers were used for amplification; then the amplified product was recovered by gel extraction to obtain the target product 246-3. There are homology arm sequences between the backbone fragment and the fragment and between the fragments, and multiple fragments can be assembled into a complete vector through Gisbon. The Rep-CAP plasmid of mutant 1 can be recombined and constructed by mixing the backbone fragment, 246-1, 246-2, and 246-3 in the following steps and proportions.

According to the Cap sequence of mutant 2, the following primers were designed, and the specific steps included: The Rep-CAP plasmid of AAV2 was used as a template and the primers Cap-f+YJ69-R were used for amplification; then the amplified product was recovered by gel extraction to obtain the target product 246-1. The Rep-CAP plasmid of AAV2 was used as a template and primers YJ69-F+YJ72-R were used for amplification; then the amplified product was recovered by gel extraction to obtain the target product 246-2. The Rep-CAP plasmid of AAV2 was used as a template and primers YJ72-F+247-R were used for amplification; then the amplified product was recovered by gel extraction to obtain the target product 247-3. The Rep-CAP plasmid of AAV2 was used as a template and primers 247-F+cap-r were used for amplification; then the amplified product was recovered by gel extraction to obtain the target product 247-4. There are homology arm sequences between the backbone fragment and the fragment and between the fragments, and multiple fragments can be assembled into a complete vector through Gisbon. The Rep-CAP plasmid of mutant 2 can be recombined and constructed by mixing the backbone fragment, 246-1, 246-2, 247-3, and 247-4 in the following steps and proportions.

According to the Cap sequence of mutant 3, the following primers were designed, and the specific steps included: The Rep-CAP plasmid of AAV2 was used as a template and the primers Cap-f+YJ69-R were used for amplification; then the amplified product was recovered by gel extraction to obtain the target product 246-1. The Rep-CAP plasmid of AAV2 was used as a template and primers YJ69-F+YJ72-R were used for amplification; then the amplified product was recovered by gel extraction to obtain the target product 246-2. The Rep-CAP plasmid of AAV2 was used as a template and primers YJ72-F+248-R were used for amplification; then the amplified product was recovered by gel extraction to obtain the target product 248-3. The Rep-CAP plasmid of AAV2 was used as a template and primers 248-F+cap-r were used for amplification; then the amplified product was recovered by gel extraction to obtain the target product 248-4. There are homology arm sequences between the backbone fragment and the fragment and between the fragments, and multiple fragments can be assembled into a complete vector through Gisbon. The Rep-CAP plasmid of mutant 3 can be recombined and constructed by mixing the backbone fragment, 246-1, 246-2, 248-3, and 248-4 in the following steps and proportions.

The primers involved in the construction of the Rep-CAP vector of mutants 1-3 of the AAV capsid protein in this application are:

| Primer | Primer |
|---|---|
| name | sequence (5′ → 3′) |
| Cap-f | CATCTTTGAACAATAAATGATTTAAATCAGGTATG |
| | (SEQ ID NO: 13) |
| cap-r | TCAACTGAAACGAATCAACCGGTTT |
| | (SEQ ID NO: 14) |
| YJ69-R | TGTTGATTCTGTCCAGAACCGTTAATAGTCTTGCT |
| | CAAGTAATACAGGTACTGGTCGAT |
| | (SEQ ID NO: 15) |
| YJ69-F | ACGGTTCTGGACAGAATCAACAAACGCTAAAATTT |
| | TCTCAGGCCGGAGCGAGT |
| | (SEQ ID NO: 16) |
| YJ72-R | GTTGTTTTGAGTCACAGTGGTTGATACTCGCTGCT |
| | GGCGGTAACAG |
| | (SEQ ID NO: 17) |
| YJ72-F | ATCAACCACTGTGACTCAAAACAACAACAGTGAAT |
| | ACTCGTGGACTGGAG |
| | (SEQ ID NO: 18) |
| 247-R | TTGTCTGTTGCCAGCCTGGAGGTTGGTAGATACAG |
| | AACCATACT |
| | (SEQ ID NO: 19) |
| 247-F | CCTCCAGGCTGGCAACAGACAAGCAGCTACCGCAG |
| | AT |
| | (SEQ ID NO: 20) |
| 248-R | CTGCTTGTCTCTGGAGGTTGGTAGATACAGAACCA |
| | TACT |
| | (SEQ ID NO: 21) |
| 248-F | CAACCTCCAGAGACAAGCAGCTACCGCAGAT |
| | (SEQ ID NO: 22) |

One clean PCR tube with a volume of 200 μL was taken, labeled, and placed on an ice box. The reaction solution was prepared with the digested backbone fragment above and each target fragment (i.e., the target product above). In the reaction solution, the molar ratio of the digested backbone fragment to each target fragment was 1:3. The reaction solution was reacted in a PCR instrument at 50° C. for 30 min to perform recombination ligation. 50 μL of competent cells were thawed on ice, and 10 μL of ligation product was mixed with DH5a competent cells. The resulting mixture was placed on ice for 20-30 min; heat-shocked at 42° C. for 45 seconds; quickly placed in an ice bath on ice for 2 min, and then added to 400 μL of SOC medium (without antibiotics) for recovery and incubated at 37° C., 200 rpm for 1 h. The incubated product was evenly spread on an Amp-resistant plate (50 μg/ml) and cultured at 37° C. for 14 hours. The monoclonal bacteria were selected and expanded in 4 mL of liquid LB medium (Amp+ resistance) for 14 hours at 37° C.

The bacterial solution was centrifuged at 12,000 rpm for 1 min and the supernatant medium was poured off to obtain an EP tube containing the bacterial precipitate. 250 μL of a mixture of buffer P1/RNaseA was added to the EP tube and the bacteria were resuspended through vortex at a high speed. Subsequently, 250 μL of buffer P2 was added to the EP tube and then the EP tube was turned up and down 8-10 times. Then, 350 μL of buffer P3 was added to the EP tube, and the solution was immediately inverted and mixed 8-10 times to allow for thorough neutralization. The EP tube was centrifuged at 13,000 rpm for 10 minutes, and the resulting supernatant was passed through the column. The centrifuge tube containing the resulting column was centrifuged at 12,000 rpm for 1 min and the waste liquid was discarded. 500 μL of PW1 was added to the centrifuge tube, the resulting centrifuge tube was centrifuged at 12,000 rpm for 1 min, and the waste liquid was discarded. 600 μL of PW2 was added to the centrifuge tube, the obtained centrifuge tube was centrifuged at 12,000 rpm for 1 min, and the supernatant was poured off. 600 μL of PW2 was added to the centrifuge tube, the resulting centrifuge tube was centrifuged at 12,000 rpm for 1 min, and the supernatant was poured off. After the centrifuge tube was centrifuged without load at 12,000 rpm for 2 min, 30-50 μL of eluent pre-heated at 55° C. was added, the centrifuge tube was allowed to stand for 2 min, and centrifuged at 12,000 rpm for 1 min. Concentration detection was performed using a micro nucleic acid quantifier.

The obtained plasmid was tested for concentration, and 10 μL of the positive plasmid identified by enzyme digestion was taken for sequencing, and the positive plasmid was stored at −20° C. The sequencing results show that the obtained plasmid could encode a mutant capsid protein VP1. Finally, according to the amount of virus required for later testing, the relevant helper plasmids, each group of Rep-Cap plasmids (AAV2, AAV9, and mutants in this application) and GOI plasmids (ssAAV.CAG.Fluc-2a-eGFP.WPRE.SV40 pA) were extracted.

### 3) Packaging and Purification of a Mutant of a Virus Serotype

The Rep-Cap plasmids of each group (AAV2, AAV9 and the mutants of AAV of the present application), a plasmid expressing firefly luciferase (Fluc) and green fluorescent protein (EGFP), and a pHelper plasmid were co-transfected into an HEK-293T cell in an appropriate amount, and then the AAV viruses were purified by iodixanol gradient ultracentrifugation. The measured virus titer was 1E+12GC/mL-1E+13GC/mL, which was a suitable titer. The obtained AAV viruses were stored at −80° C. for future use.

### Example 2: Comparative Test of Various Indicators of Mutant Serotypes

### 1) Animal Injection and Dissection

Animal experiments were performed using 6-8 week old C57 male mice. The relevant viruses were formulated according to the designed experimental and control groups. Each group was injected with 1E+12GC viruses per mouse. Animal dissection and sampling of various organs were performed 4 weeks after injection. Samples were snap frozen in liquid nitrogen immediately after sampling and used for subsequent experiments such as RNA extraction and western blot (WB) detection, respectively.

2) Detection of mRNA Expression Levels of Target Genes

### 2.1) Total RNA Extraction and Reverse Transcription:

Sample grinding: The grinder was pre-cooled 10 min in advance and the grinding parameters were set. An animal tissue sample stored in a −80° C. refrigerator was taken out. About 50-100 mg of the tissue was taken from the sample, clipped to the size of soybean grains in a sterile petri dish, then transferred to a 1.5 mL RNase-free EP tube. At the ratio of 1 mL of TransZol Up per 50-100 mg of the tissue, an appropriate amount of TransZol Up was added to the EP tube, two clean and sterile steel grinding balls with a diameter of 3 mm were then added, and the tube was sealed with parafilm. The sample was placed and leveled in a 24-well grinding adapter, the screw was tightened, and the cap-off button was pressed. The grinding program was initiated, after the program was finished, the sample was taken out and the particle size of the sample after grinding was observed; if no large piece of the tissue remained, the subsequent extraction operation could be carried out. After grinding, the sample was centrifuged at 12,000×g for 2 min at 4° C., and the supernatant was transferred to a new RNase-free 1.5-mL eppendorf (EP) tube labeled accordingly.

Extraction of total RNA from a sample: For details, refer to the instruction manual of TransZol Up Plus RNA Kit (Beijing TransGen Biotech Co., Ltd., Item No.: ER501). Whenever 1 ml of TranZol Up was used, 0.2 mL of the RNA extraction agent was added to the sample and the resulting mixture was shaken vigorously for 5 min; then centrifugation was performed at 12,000×g and 4° C. for 10 min. At this point, the sample was divided into three layers, and the colorless aqueous phase was transferred to a new RNase-free 1.5-mL EP tube, to which an equal volume of anhydrous ethanol was added (precipitation may occur at this point), and the EP tube was gently inverted to mix evenly. The obtained solution together with the precipitate was added to the spin column, and the spin column was centrifuged at 12,000×g for 30 seconds at room temperature, and the filtrate was discarded. 500 μL of CB9 was added to the spin column, which was then centrifuged at 12,000×g for 30 seconds at room temperature, and the filtrate was discarded; the previous step was repeated once. 500 μL of WB9 was added to the spin column, which was then centrifuged at 12,000×g for 30 seconds at room temperature, and the filtrate was discarded; the previous step was repeated once. The spin column was centrifuged at 12,000×g for 2 min at room temperature to completely remove residual ethanol. The spin column was placed into a RNase-free 1.5-mL EP tube and 30-50 μL (depending on tissue size) of RNase-free water was added to the center of the spin column and allowed to stand for 1 min at room temperature. The EP tube containing the spin column was centrifuged at 12,000×g for 1 min at room temperature to elute RNA.

Determination of nucleic acid concentration in the sample: The RNA concentration was detected using the micro nucleic acid quantifier; the concentration, OD260/280, and OD260/230 were recorded; and the RNA was stored at −80° C.

Reverse transcription: EasyScript® All-in-One First-Strand cDNA Synthesis SuperMix for qPCR (One-Step gDNA Removal) (Beijing TransGen Biotech Co., Ltd., Item No.: AE341-03) was used for each group of RNA samples, and the specific steps were described in the instruction manual.

2.2) Quantitative PCR (qPCR) Experiments:

Each group of cDNA was used as a template and the qPCR system was prepared according to the instructions of 2× SYBR Green qPCR Master Mix (BIMAKE, Item No.: B21203):

qPCR system

| Reagent | Usage | |
|---|---|---|
| 2× SYBR Green qPCR Master Mix | 10 | μL |
| cDNA template | 1.5 | μL |
| Upstream primer (10 μM) | 1 | μL |
| Downstream primer (10 μM) | 1 | μL |
| ROX Reference Dye | 0.4 | μL |

| | |
|---|---|
| Deionized water | Up to 20 μL |

| | Primer sequence |
|---|---|
| Primer name | (5′->3′) |
| Fluc2-qPCR-F1 | AACCAGCGCCATTCTGATCA |
| | (SEQ ID NO: 23) |
| Fluc2-qPCR-R1 | TCGGGGTTGTTAACGTAGCC |
| | (SEQ ID NO: 24) |
| GAPDH-F2 | CAGGAGAGTGTTTCCTCGTCC |
| | (SEQ ID NO: 25) |
| GAPDH-R2 | TTCCCATTCTCGGCCTTGAC |
| | (SEQ ID NO: 26) |

Settings of qPCR procedure

| | 1 | | | | | | | | | | | |
|---|---|---|---|---|---|---|---|---|---|---|---|---|
| | Hot-Start | | | | | | | | | | | |
| | DNA | | 2 | | | | | | | | | |
| | Polymerase | | PCR | | | | 3 | | | | | |
| | Activation | | Cycle (40 cycles) | | | | Melt Curve | | | | | |
| Step | Hold | | Denature | | Anneal/Extend | | Cycle (1 cycle) | | | | | |
| Temp | 95.0° | C. | 95.0° | C. | 60.0° | C. | 95.0° | C. | 60.0° | C. | 95° | C. |
| Time | 10 | min | 15 | sec | 30 | sec | 15 | sec | 60 | sec | 15 | sec |

### 2.3) Data Analysis

Relative expression was calculated from the Ct values of each group according to the formula 2\hat{ }-ΔΔct.

### 3) WB Detection of Expression Levels of Target Proteins

Sample pre-treatment: The tissue was cut into tiny pieces and weighed. The weight of the tissue was recorded. The tiny pieces were placed in a 1.5 mL or 2 mL centrifuge tube; and the centrifuge tube was labeled and frozen at −80° C. for use. The cryo-grinder was pre-chilled. RIPA (Shanghai Beyotime Biotechnology Co., Ltd., P0013B) lysate was solubilized (Phenylmethylsulfonyl fluoride (PMSF) was added within a few minutes prior to use to obtain a final PMSF concentration of 1 mM). The above complete lysate was added to the centrifuge tube at a ratio of 150-250 μL of lysate per 20 mg of the tissue. Then two sterilized zirconium oxide grinding balls were added to the centrifuge tube and the sample was ground directly in the lysis buffer (the tissue sample of the brain or spinal cord: The temperature was −20° C., and the frequency was 70 Hz; the oscillation time was 50 seconds followed by a pause of 10 seconds, and this cycle was repeated 3-4 times; and the sample of the muscle or liver: The temperature was −20° C., and the frequency was 70 Hz; the oscillation time was 50 seconds followed by a pause of 10 seconds, and this cycle was repeated 5-7 times). After grinding, the sample was centrifuged in a freezing centrifuge at 4° C., 12,000×g for 5-10 min, and then the supernatant was transferred to a new sterilized EP tube and stored at −20° C. or −80° C.

Determination of protein concentration: Protein concentration was determined according to the method in the protein concentration assay kit based on the modified BCA method (Sangon Biotech (Shanghai) Co., Ltd., Item No.: C503051). Then, an appropriate amount of a sample of homogenate protein was taken according to the required amount, mixed with the corresponding amount of 5× SDS-PAGE protein loading buffer, boiled in a water bath for 10 min, cooled and centrifuged at a low speed for a while, and waited for sample loading.

### Western Blot (WB) Detection:

SDS-PAGE electrophoresis: The appropriate loading amount was determined by the protein concentration and expression level, and was less than 20 μL/well. The loading amount of the tissue homogenate protein was about 20-50 μg. The specific operation process of electrophoresis was as follows: The comb on the precast gel was pulled out and the gel was mounted in the electrophoresis chamber. The electrophoresis buffer was added to the inner and outer chambers, where the inner chamber was filled with a freshly prepared buffer and checked for leaks. If there was no leakage in the inner chamber, then electrophoresis buffer could be added to the outer chamber. An appropriate amount of a treated protein sample was taken for loading, the pre-stained standard protein was used as a reference, and electrophoresis was carried out on a Tanon (Shanghai tanon life science Co., Ltd.) electrophoresis device at a constant voltage of 100 V for 100 min until the bromophenol blue reached the bottom of the gel. The power was turned off, the precast gel plate was carefully unloaded, and the gel was removed and placed in the transfer buffer to await subsequent manipulation.

Transfer: Six sheets of filter paper and one sheet of PVDF membrane were cut according to the gel area. The PVDF membrane was soaked in methanol for 5-10 seconds and then transferred to and soaked in the transfer buffer for 5 min, and the filter paper was also pre-wetted in the transfer buffer. Installation of the transfer device: Negative electrode (black plate)—sponge—3 layers of moistened filter paper—gel—PVDF membrane—3 layers of moistened filter paper—sponge—positive electrode (transparent plate). Each layer of air bubbles in the transfer device was driven away to avoid affecting the transfer effect, and after clamping the bracket, the transfer device was placed into the electrotransfer tank. The membrane was transferred in an ice bath at a constant voltage of 100 V for 100 min. The success of membrane transfer was determined by whether protein bands of the pre-stained molecular weight marker were completely transferred to the PVDF membrane. The transferred PVDF membrane was soaked and washed in PBST solution at room temperature for 5 min, and the PVDF membrane was cut according to the requirements. Be careful not to let the PVDF membrane dry out during the cutting process.

Blocking and antibody incubation: The PVDF membrane was incubated with a blocking solution (5% skimmed milk powder) for 2 hours at room temperature or overnight at 4° C. The blocked PVDF membrane was transferred to a primary antibody hybridization solution and incubated for 1 h at room temperature or overnight at 4° C. Luciferase Rabbit Polyclonal antibody (Proteintech Group, Inc., 27986-1-AP) was added to 4 mL of QuickBlock™ Western primary antibody dilution (Shanghai beyotime biotechnology Co., Ltd., P0256) in a ratio of 1:2000 to prepare the primary antibody hybridization; GADPH Rabbit Polyclonal antibody (Proteintech Group, Inc., 10494-1-AP) was added to 4 mL of QuickBlock™ Western primary antibody dilution (Shanghai beyotime biotechnology Co., Ltd., P0256) in a ratio of 1:2000 to prepare the primary antibody hybridization; and Rabbit GFP tag Polyclonal antibody (Proteintech Group, Inc., 50430-2-AP) was added to 4 mL of QuickBlock™ Western primary antibody dilution (Shanghai beyotime biotechnology Co., Ltd., P0256) in a ratio of 1:2000 to prepare the primary antibody hybridization. Then the membrane was washed three times with PBST for 5 min each time. The washed PVDF membrane was transferred into the secondary antibody hybridization solution and incubated at room temperature for 1 h. HRP-conjugated Affinipure Goat Anti-Rabbit IgG (H+L) (Proteintech Group, Inc., SA00001-2) was added to 4 mL of QuickBlock™ Western Secondary Antibody Diluent (Shanghai beyotime biotechnology Co., Ltd., P0258) at a ratio of 1:5000 to obtain the secondary antibody hybridization solution. The membrane was washed three times with PBST for 5 min each time.

Color development: ECL chemiluminescence kit's liquid A and liquid B were mixed in an equal volume. After shaking and mixing, the luminescent liquid was dropped on the PVDF membrane such that all the PVDF membrane was covered with the luminescent liquid, and the exposure time was adjusted such that the protein bands were clear, and then photographed by the instrument.

### 4) Results:

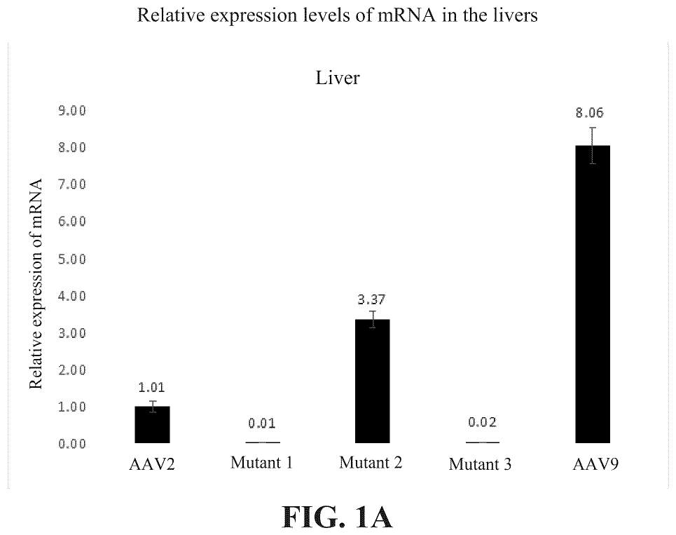

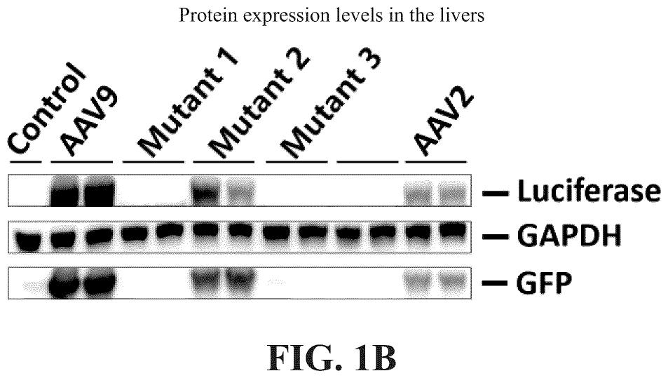

Comparing the above different modified mutants with AAV2 and AAV9 controls, it can be found that the three modified mutants have the characteristics of low liver tropism. The liver mRNA levels of mutant 1, mutant 2, and mutant 3 (FIG. 1A) are 806-fold, 2.39-fold, and 403-fold lower than that of AAV9, respectively. The liver mRNA levels of mutants 1 and 3 are 101-fold and 50.5-fold lower than that of AAV2. Detection of target protein levels (firefly luciferase and eGFP) (FIG. 1B) also confirmed the characteristic of low liver tropism. In addition, we have also detected and analyzed the targeting to different tissues and organs.

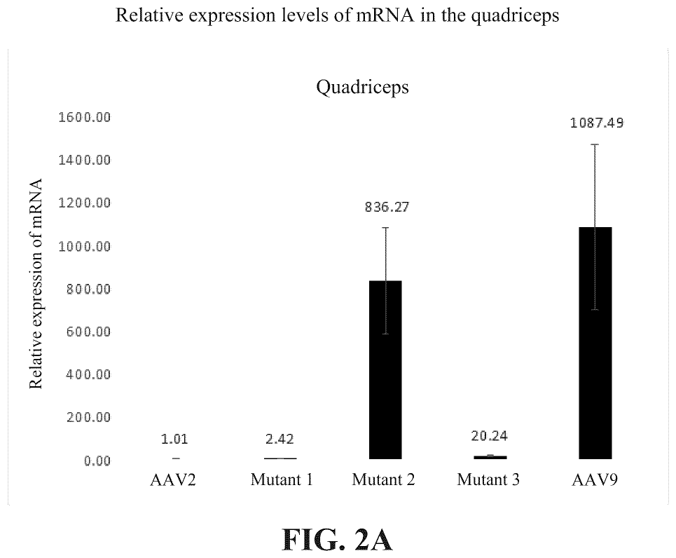

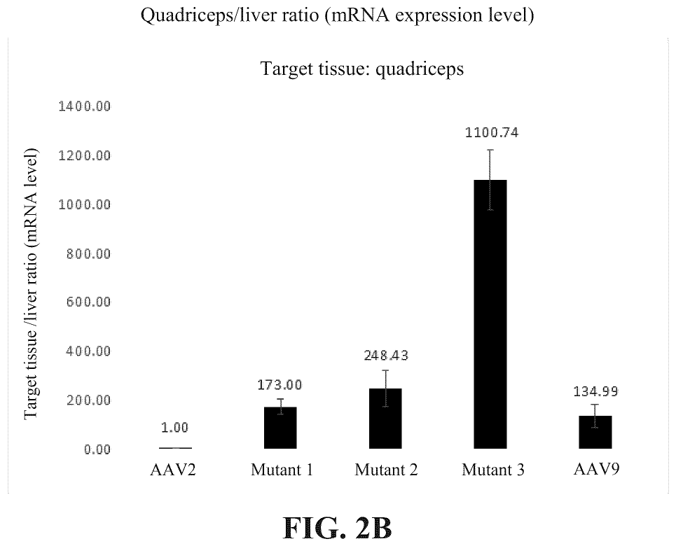

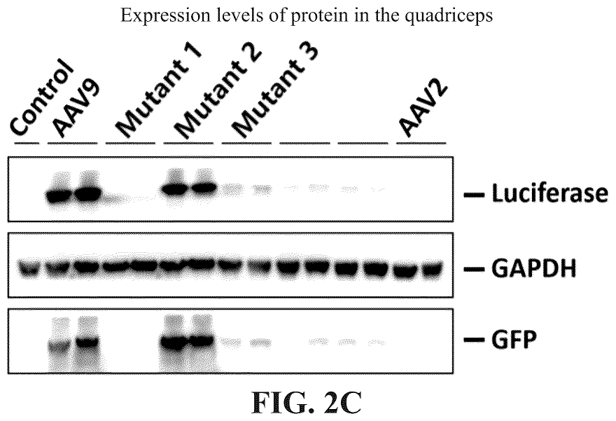

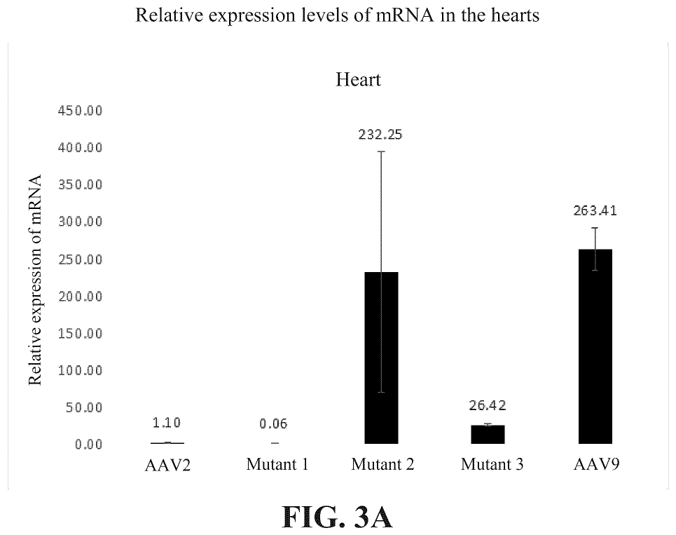

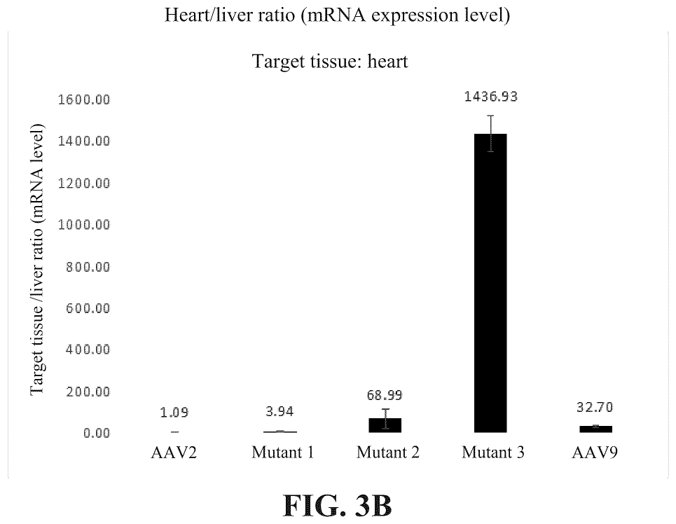

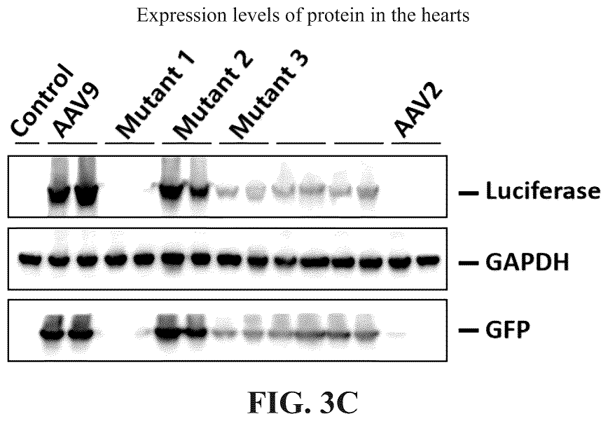

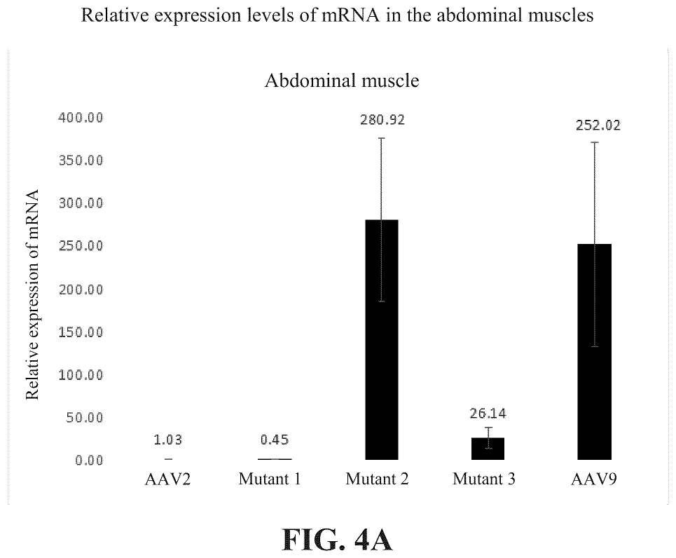

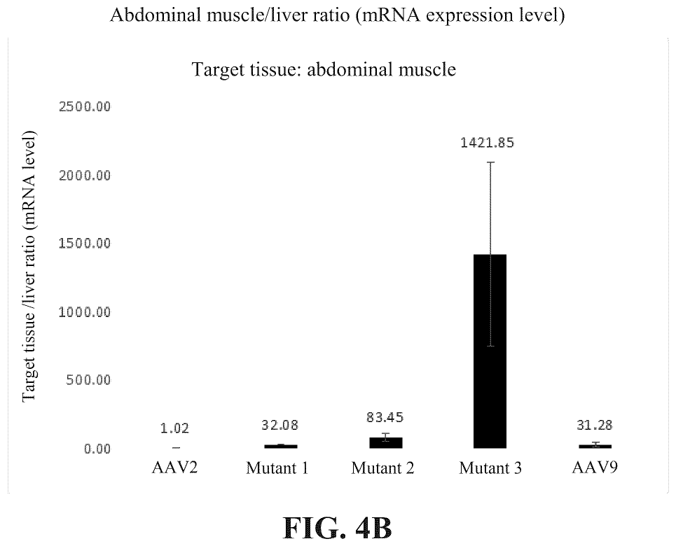

For the targeting to muscle and heart, mutant 2 and mutant 3 have higher infectious ability to quadriceps, abdominal muscles, and hearts than AAV2 (836-fold and 20-fold for quadriceps (FIG. 2A), 280-fold and 26-fold for abdominal muscles (FIG. 4A), and 232-fold and 26-fold for hearts (FIG. 3A) regarding the comparison of mRNA levels; regarding the comparison of protein levels, mutants 2 and 3 have higher levels than AAV2 in both quadriceps (FIG. 2C) and hearts (FIG. 3C)). Moreover, mutant 2 is even closer to AAV9 in terms of mRNA and protein levels (FIG. 2C), and the ratio of mRNA and protein levels in the target tissues to those in the liver of mutant 2 is even higher than that of AAV9 (1.8-fold for quadriceps (FIG. 2B), 2.6-fold for abdominal muscles (FIG. 4B), and 2.1-fold for hearts (FIG. 3B)).

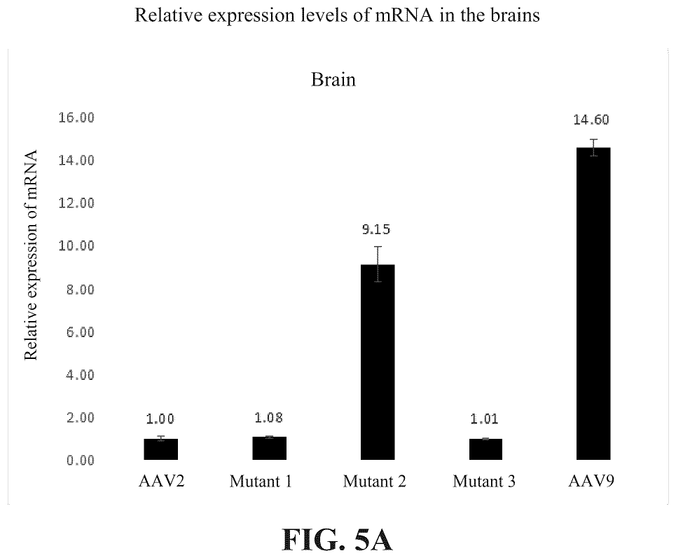

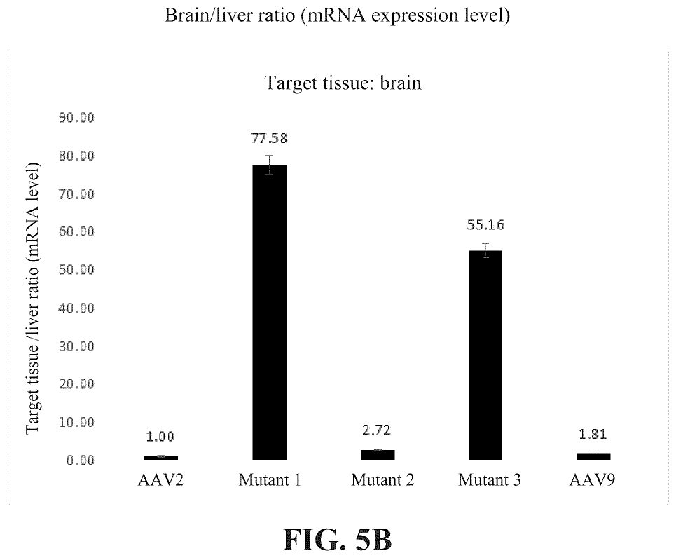

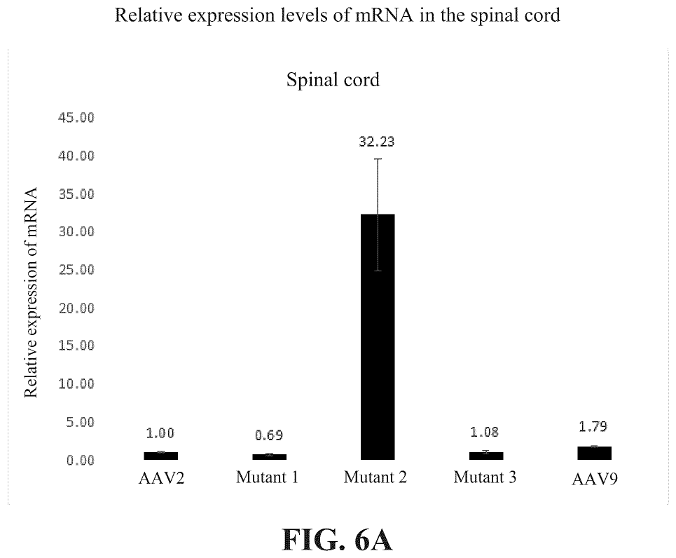

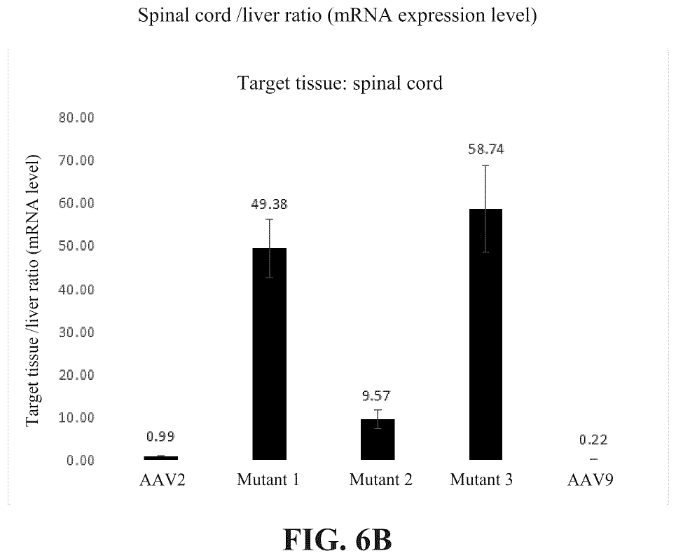

For the targeting to brain and spinal cord, mutant 2 shows a better targeting advantage over mutants 1 and 3. Compared to AAV2, mutant 2 has a 9-fold and 32-fold higher ability to infect the brain (FIG. 5A) and spinal cord (FIG. 6A), respectively. Mutant 2 is even more infectious to the spinal cord than AAV9. Among them, the ratio of mRNA level in target tissues to mRNA level in liver shows its unique advantages. For example, the ratio of mRNA levels in the brain to mRNA levels in the liver of mutant 2 is 1.5 times that of AAV9 (FIG. 5B), and the ratio of mRNA levels in the spinal cord to mRNA levels in the liver was 43.5 times (FIG. 6B). It can be seen that the specificity advantage of mutant 2 is very obvious. In addition, it is worth noting that mutants 1 and 3 have a weak ability to infect the brain and spinal cord (even though the ratio of mRNA and protein levels in their target tissues to mRNA and protein levels in the liver is high).

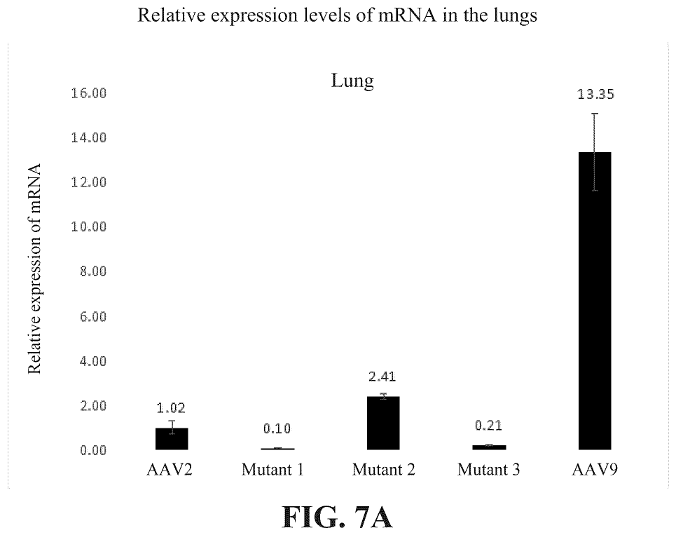

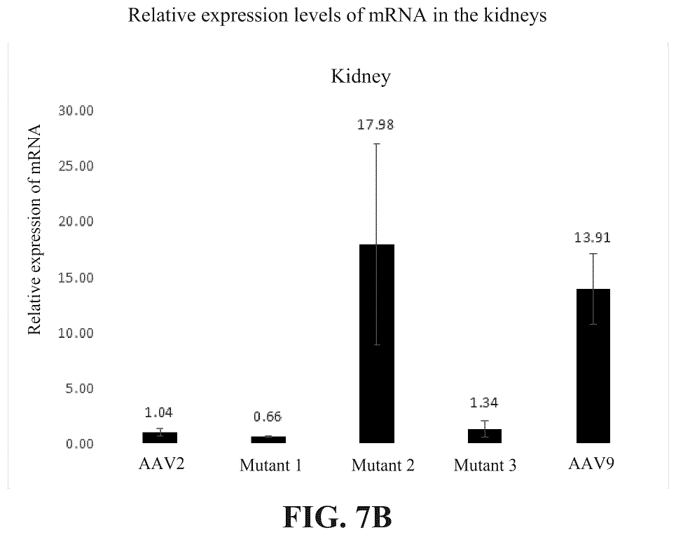

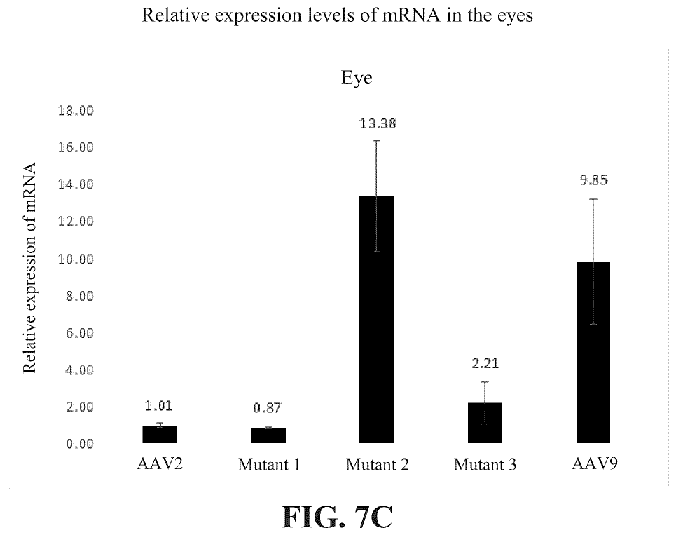

For the targeting to lung, kidney, and eye, mutant 2 has slightly higher infectivity to the kidney and eye than AAV9, except that the ability to infect the lung is about 5.5 times lower than that of AAV9. Moreover, compared to AAV2, the targeting ability of mutant 2 is approximately 2.4-fold (FIG. 7A, lung), 18-fold (FIG. 7B, kidney), and 13-fold (FIG. 7C, eye) of that of AAV2, respectively.

Based on the above results, it is proved that the three mutants with the same backbone skeletal sequence have the advantage of low targeting to liver, but shows different characteristics due to the difference of a few amino acids in the ring VIII of CAP protein. For example, the liver tropism of mutant 1 is 101 times lower than that of AAV2, but mutant 1 also has low targeting to various organs. Since mutant 1 is consistent with AAV2 except for the AAV9 sequence replacement at a specific site, it is fully confirmed that the substituted sequence (i.e., the backbone sequence) can greatly reduce the effect of liver tropism, which is also a new discovery of this application. Mutant 2 includes a further mutation on the base of mutant 1 (AAV2: R585A), resulting in targeting to muscle and heart comparable to AAV9 and better targeting to spinal cord. In addition to its very low liver tropism, mutant 3 has a lower ability than AAV9 and a stronger ability than AAV2 in terms of targeting muscle and heart, and has low targeting to other organs. Mutant 3 has a high specific targeting to muscle and heart.

Therefore, in this application, three “chimeras” of AAV serotypes with low liver tropism were constructed through analytical design and “loop swapping” technology, which possesses some advantages of both AAV2 and AAV9. In addition to the characteristics of targeting different organs displayed by the three mutants themselves, based on their excellent target organ/liver ratios (i.e., the ratio of mRNA and/or protein levels in the target organs to those in the liver), they can still be used as a “universal” modified backbone to insert specific sequences such as targeted peptides and antibodies at specific sites to enhance their characteristics of targeting specific tissues but retaining the lower liver tropism, so as to achieve better specificity, avoid the problem of hepatotoxicity in current clinical trials, and provide safer and more reliable gene therapy products for the majority of patients. In this application, the “loop swapping” technique and the rational design analysis method based on AAV structure were used to make use of the complementary advantages of AAV2 and AAV9, with the aim of reconstructing an AAV serotype having low liver tropism and good targeting to specific tissues. In addition, substitution of a sequence segment on the surface of the cap structure (e.g., a ring with a protruding surface) is also a simple and effective way to avoid the influence of a pre-stored neutralizing antibody on the AAV parent in vivo, and we will further explore it in the future.

Finally, it should be noted that the above embodiments are only used to illustrate the technical solution of the present application and not to limit the scope of protection of the present application. Although the present application is described in detail with reference to the preferred embodiments, it should be understood by those of ordinary skill in the art that modifications or equivalent substitutions may be made to the technical solutions of the present application without departing from the substance and scope of the technical solutions of the present application.

Amino acid and nucleotide sequences referred to herein include the following.

(amino acid sequence of wild-type AAV2 VP1)
SEQ ID NO: 1
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPAA
PSGLGTNTMATGSGAPMADNNEGADGVGNSSGNWHCDSTWMGDRVITTSTRTWALPTYNNHLYKQ
ISSQSGASNDNHYFGYSTPWGYFDFNRFHCHFSPRDWQRLINNNWGFRPKRLNFKLFNIQVKEVTQN
DGTTTIANNLTSTVQVFTDSEYQLPYVLGSAHQGCLPPFPADVFMVPQYGYLTLNNGSQAVGRSSFYC
LEYFPSQMLRTGNNFTFSYTFEDVPFHSSYAHSQSLDRLMNPLIDQYLYYLSRTNTPSGTTTQSRLQFS
QAGASDIRDQSRNWLPGPCYRQQRVSKTSADNNNSEYSWTGATKYHLNGRDSLVNPGPAMASHKD
DEEKFFPQSGVLIFGKQGSEKTNVDIEKVMITDEEEIRTTNPVATEQYGSVSTNLQRGNRQAATADVNT
QGVLPGMVWQDRDVYLQGPIWAKIPHTDGHFHPSPLMGGFGLKHPPPQILIKNTPVPANPSTTFSAAK
FASFITQYSTGQVSVEIEWELQKENSKRWNPEIQYTSNYNKSVNVDFTVDTNGVYSEPRPIGTRYLTR
NL*
(substitution sequence)
SEQ ID NO: 2
KTINGSGQNQQTLK
(substitution sequence)
SEQ ID NO: 3
TTVTQ
(substituted sequence)
SEQ ID NO: 4
RTNTPSGTTTQSRLQ
(substituted sequence)
SEQ ID NO: 5
KTSAD
(VP1 amino acid sequence of mutant 1)
SEQ ID NO: 6
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPAA
PSGLGTNTMATGSGAPMADNNEGADGVGNSSGNWHCDSTWMGDRVITTSTRTWALPTYNNHLYKQ
ISSQSGASNDNHYFGYSTPWGYFDFNRFHCHFSPRDWQRLINNNWGFRPKRLNFKLFNIQVKEVTQN
DGTTTIANNLTSTVQVFTDSEYQLPYVLGSAHQGCLPPFPADVFMVPQYGYLTLNNGSQAVGRSSFYC
LEYFPSQMLRTGNNFTFSYTFEDVPFHSSYAHSQSLDRLMNPLIDQYLYYLSKTINGSGQNQQTLKFSQ
AGASDIRDQSRNWLPGPCYRQQRVSTTVTQNNNSEYSWTGATKYHLNGRDSLVNPGPAMASHKDDE
EKFFPQSGVLIFGKQGSEKTNVDIEKVMITDEEEIRTTNPVATEQYGSVSTNLQRGNRQAATADVNTQ
GVLPGMVWQDRDVYLQGPIWAKIPHTDGHFHPSPLMGGFGLKHPPPQILIKNTPVPANPSTTFSAAKF
ASFITQYSTGQVSVEIEWELQKENSKRWNPEIQYTSNYNKSVNVDFTVDTNGVYSEPRPIGTRYLTRN
L*
(VP1 amino acid sequence of mutant 2)
SEQ ID NO: 7
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPAA
PSGLGTNTMATGSGAPMADNNEGADGVGNSSGNWHCDSTWMGDRVITTSTRTWALPTYNNHLYKQ
ISSQSGASNDNHYFGYSTPWGYFDFNRFHCHFSPRDWQRLINNNWGFRPKRLNFKLFNIQVKEVTQN
DGTTTIANNLTSTVQVFTDSEYQLPYVLGSAHQGCLPPFPADVFMVPQYGYLTLNNGSQAVGRSSFYC
LEYFPSQMLRTGNNFTFSYTFEDVPFHSSYAHSQSLDRLMNPLIDQYLYYLSKTINGSGQNQQTLKFSQ
AGASDIRDQSRNWLPGPCYRQQRVSTTVTQNNNSEYSWTGATKYHLNGRDSLVNPGPAMASHKDDE
EKFFPQSGVLIFGKQGSEKTNVDIEKVMITDEEEIRTTNPVATEQYGSVSTNLQAGNRQAATADVNTQ
GVLPGMVWQDRDVYLQGPIWAKIPHTDGHFHPSPLMGGFGLKHPPPQILIKNTPVPANPSTTFSAAKF
ASFITQYSTGQVSVEIEWELQKENSKRWNPEIQYTSNYNKSVNVDFTVDTNGVYSEPRPIGTRYLTRN
L*
(VP1 amino acid sequence of mutant 3)
SEQ ID NO: 8
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPAA
PSGLGTNTMATGSGAPMADNNEGADGVGNSSGNWHCDSTWMGDRVITTSTRTWALPTYNNHLYKQ
ISSQSGASNDNHYFGYSTPWGYFDFNRFHCHFSPRDWQRLINNNWGFRPKRLNFKLFNIQVKEVTQN
DGTTTIANNLTSTVQVFTDSEYQLPYVLGSAHQGCLPPFPADVFMVPQYGYLTLNNGSQAVGRSSFYC
LEYFPSQMLRTGNNFTFSYTFEDVPFHSSYAHSQSLDRLMNPLIDQYLYYLSKTINGSGQNQQTLKFSQ
AGASDIRDQSRNWLPGPCYRQQRVSTTVTQNNNSEYSWTGATKYHLNGRDSLVNPGPAMASHKDDE
EKFFPQSGVLIFGKQGSEKTNVDIEKVMITDEEEIRTTNPVATEQYGSVSTNLQRQAATADVNTQGVLP
GMVWQDRDVYLQGPIWAKIPHTDGHFHPSPLMGGFGLKHPPPQILIKNTPVPANPSTTFSAAKFASFIT
QYSTGQVSVEIEWELQKENSKRWNPEIQYTSNYNKSVNVDFTVDTNGVYSEPRPIGTRYLTRNL*
(VP1 nucleic acid sequence of AAV2 (5′->3′'))
SEQ ID NO: 9
ATGGCTGCCGATGGTTATCTTCCAGATTGGCTCGAGGACACTCTCTCTGAAGGAATAAGAC
AGTGGTGGAAGCTCAAACCTGGCCCACCACCACCAAAGCCCGCAGAGCGGCATAAGGACGACAG
CAGGGGTCTTGTGCTTCCTGGGTACAAGTACCTCGGACCCTTCAACGGACTCGACAAGGGAGAGC
CGGTCAACGAGGCAGACGCCGCGGCCCTCGAGCACGACAAAGCCTACGACCGGCAGCTCGACA
GCGGAGACAACCCGTACCTCAAGTACAACCACGCCGACGCGGAGTTTCAGGAGCGCCTTAAAGA
AGATACGTCTTTTGGGGGCAACCTCGGACGAGCAGTCTTCCAGGCGAAAAAGAGGGTTCTTGAA
CCTCTGGGCCTGGTTGAGGAACCTGTTAAGACGGCTCCGGGAAAAAAGAGGCCGGTAGAGCACT
CTCCTGTGGAGCCAGACTCCTCCTCGGGAACCGGAAAGGCGGGCCAGCAGCCTGCAAGAAAAAG
ATTGAATTTTGGTCAGACTGGAGACGCAGACTCAGTACCTGACCCCCAGCCTCTCGGACAGCCAC
CAGCAGCCCCCTCTGGTCTGGGAACTAATACGATGGCTACAGGCAGTGGCGCACCAATGGCAGAC
AATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCT
ACAAACAAATTTCCAGCCAATCAGGAGCCTCGAACGACAATCACTACTTTGGCTACAGCACCCCT
TGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGA
GGTCACGCAGAATGACGGTACGACGACGATTGCCAATAACCTTACCAGCACGGTTCAGGTGTTTA
CTGACTCGGAGTACCAGCTCCCGTACGTCCTCGGCTCGGCGCATCAAGGATGCCTCCCGCCGTTCC
CAGCAGACGTCTTCATGGTGCCACAGTATGGATACCTCACCCTGAACAACGGGAGTCAGGCAGTA
GGACGCTCTTCATTTTACTGCCTGGAGTACTTTCCTTCTCAGATGCTGCGTACCGGAAACAACTTT
ACCTTCAGCTACACTTTTGAGGACGTTCCTTTCCACAGCAGCTACGCTCACAGCCAGAGTCTGGA
CCGTCTCATGAATCCTCTCATCGACCAGTACCTGTATTACTTGAGCAGAACAAACACTCCAAGTGG
AACCACCACGCAGTCAAGGCTTCAGTTTTCTCAGGCCGGAGCGAGTGACATTCGGGACCAGTCTA
GGAACTGGCTTCCTGGACCCTGTTACCGCCAGCAGCGAGTATCAAAGACATCTGCGGATAACAAC
AACAGTGAATACTCGTGGACTGGAGCTACCAAGTACCACCTCAATGGCAGAGACTCTCTGGTGAA
TCCGGGCCCGGCCATGGCAAGCCACAAGGACGATGAAGAAAAGTTTTTTCCTCAGAGCGGGGTT
CTCATCTTTGGGAAGCAAGGCTCAGAGAAAACAAATGTGGACATTGAAAAGGTCATGATTACAGA
CGAAGAGGAAATCAGGACAACCAATCCCGTGGCTACGGAGCAGTATGGTTCTGTATCTACCAACC
TCCAGAGAGGCAACAGACAAGCAGCTACCGCAGATGTCAACACACAAGGCGTTCTTCCAGGCAT
GGTCTGGCAGGACAGAGATGTGTACCTTCAGGGGCCCATCTGGGCAAAGATTCCACACACGGAC
GGACATTTTCACCCCTCTCCCCTCATGGGTGGATTCGGACTTAAACACCCTCCTCCACAGATTCTC
ATCAAGAACACCCCGGTACCTGCGAATCCTTCGACCACCTTCAGTGCGGCAAAGTTTGCTTCCTTC
ATCACACAGTACTCCACGGGACAGGTCAGCGTGGAGATCGAGTGGGAGCTGCAGAAGGAAAACA
GCAAACGCTGGAATCCCGAAATTCAGTACACTTCCAACTACAACAAGTCTGTTAATGTGGACTTTA
CTGTGGACACTAATGGCGTGTATTCAGAGCCTCGCCCCATTGGCACCAGATACCTGACTCGTAATC
TGTAA
(VP1 nucleic acid sequence of mutant 1 (5′->3′))
SEQ ID NO: 10
ATGGCTGCCGATGGTTATCTTCCAGATTGGCTCGAGGACACTCTCTCTGAAGGAATAAGAC
AGTGGTGGAAGCTCAAACCTGGCCCACCACCACCAAAGCCCGCAGAGCGGCATAAGGACGACAG
CAGGGGTCTTGTGCTTCCTGGGTACAAGTACCTCGGACCCTTCAACGGACTCGACAAGGGAGAGC
CGGTCAACGAGGCAGACGCCGCGGCCCTCGAGCACGACAAAGCCTACGACCGGCAGCTCGACA
GCGGAGACAACCCGTACCTCAAGTACAACCACGCCGACGCGGAGTTTCAGGAGCGCCTTAAAGA
AGATACGTCTTTTGGGGGCAACCTCGGACGAGCAGTCTTCCAGGCGAAAAAGAGGGTTCTTGAA
CCTCTGGGCCTGGTTGAGGAACCTGTTAAGACGGCTCCGGGAAAAAAGAGGCCGGTAGAGCACT
CTCCTGTGGAGCCAGACTCCTCCTCGGGAACCGGAAAGGCGGGCCAGCAGCCTGCAAGAAAAAG
ATTGAATTTTGGTCAGACTGGAGACGCAGACTCAGTACCTGACCCCCAGCCTCTCGGACAGCCAC
CAGCAGCCCCCTCTGGTCTGGGAACTAATACGATGGCTACAGGCAGTGGCGCACCAATGGCAGAC
AATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCT
ACAAACAAATTTCCAGCCAATCAGGAGCCTCGAACGACAATCACTACTTTGGCTACAGCACCCCT
TGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGA
GGTCACGCAGAATGACGGTACGACGACGATTGCCAATAACCTTACCAGCACGGTTCAGGTGTTTA
CTGACTCGGAGTACCAGCTCCCGTACGTCCTCGGCTCGGCGCATCAAGGATGCCTCCCGCCGTTCC
CAGCAGACGTCTTCATGGTGCCACAGTATGGATACCTCACCCTGAACAACGGGAGTCAGGCAGTA
GGACGCTCTTCATTTTACTGCCTGGAGTACTTTCCTTCTCAGATGCTGCGTACCGGAAACAACTTT
ACCTTCAGCTACACTTTTGAGGACGTTCCTTTCCACAGCAGCTACGCTCACAGCCAGAGTCTGGA
CCGTCTCATGAATCCTCTCATCGACCAGTACCTGTATTACTTGAGCAAGACTATTAACGGTTCTGGA
CAGAATCAACAAACGCTAAAATTTTCTCAGGCCGGAGCGAGTGACATTCGGGACCAGTCTAGGAA
CTGGCTTCCTGGACCCTGTTACCGCCAGCAGCGAGTATCAACCACTGTGACTCAAAACAACAACA
GTGAATACTCGTGGACTGGAGCTACCAAGTACCACCTCAATGGCAGAGACTCTCTGGTGAATCCG
GGCCCGGCCATGGCAAGCCACAAGGACGATGAAGAAAAGTTTTTTCCTCAGAGCGGGGTTCTCAT
CTTTGGGAAGCAAGGCTCAGAGAAAACAAATGTGGACATTGAAAAGGTCATGATTACAGACGAA
GAGGAAATCAGGACAACCAATCCCGTGGCTACGGAGCAGTATGGTTCTGTATCTACCAACCTCCA
GAGAGGCAACAGACAAGCAGCTACCGCAGATGTCAACACACAAGGCGTTCTTCCAGGCATGGTC
TGGCAGGACAGAGATGTGTACCTTCAGGGGCCCATCTGGGCAAAGATTCCACACACGGACGGAC
ATTTTCACCCCTCTCCCCTCATGGGTGGATTCGGACTTAAACACCCTCCTCCACAGATTCTCATCAA
GAACACCCCGGTACCTGCGAATCCTTCGACCACCTTCAGTGCGGCAAAGTTTGCTTCCTTCATCAC
ACAGTACTCCACGGGACAGGTCAGCGTGGAGATCGAGTGGGAGCTGCAGAAGGAAAACAGCAA
ACGCTGGAATCCCGAAATTCAGTACACTTCCAACTACAACAAGTCTGTTAATGTGGACTTTACTGT
GGACACTAATGGCGTGTATTCAGAGCCTCGCCCCATTGGCACCAGATACCTGACTCGTAATCTGTA
A
(VP1 nucleic acid sequence of mutant 2 (5′->3′))
SEQ ID NO: 11
ATGGCTGCCGATGGTTATCTTCCAGATTGGCTCGAGGACACTCTCTCTGAAGGAATAAGAC
AGTGGTGGAAGCTCAAACCTGGCCCACCACCACCAAAGCCCGCAGAGCGGCATAAGGACGACAG
CAGGGGTCTTGTGCTTCCTGGGTACAAGTACCTCGGACCCTTCAACGGACTCGACAAGGGAGAGC
CGGTCAACGAGGCAGACGCCGCGGCCCTCGAGCACGACAAAGCCTACGACCGGCAGCTCGACA
GCGGAGACAACCCGTACCTCAAGTACAACCACGCCGACGCGGAGTTTCAGGAGCGCCTTAAAGA
AGATACGTCTTTTGGGGGCAACCTCGGACGAGCAGTCTTCCAGGCGAAAAAGAGGGTTCTTGAA
CCTCTGGGCCTGGTTGAGGAACCTGTTAAGACGGCTCCGGGAAAAAAGAGGCCGGTAGAGCACT
CTCCTGTGGAGCCAGACTCCTCCTCGGGAACCGGAAAGGCGGGCCAGCAGCCTGCAAGAAAAAG
ATTGAATTTTGGTCAGACTGGAGACGCAGACTCAGTACCTGACCCCCAGCCTCTCGGACAGCCAC
CAGCAGCCCCCTCTGGTCTGGGAACTAATACGATGGCTACAGGCAGTGGCGCACCAATGGCAGAC
AATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCT
ACAAACAAATTTCCAGCCAATCAGGAGCCTCGAACGACAATCACTACTTTGGCTACAGCACCCCT
TGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGA
GGTCACGCAGAATGACGGTACGACGACGATTGCCAATAACCTTACCAGCACGGTTCAGGTGTTTA
CTGACTCGGAGTACCAGCTCCCGTACGTCCTCGGCTCGGCGCATCAAGGATGCCTCCCGCCGTTCC
CAGCAGACGTCTTCATGGTGCCACAGTATGGATACCTCACCCTGAACAACGGGAGTCAGGCAGTA
GGACGCTCTTCATTTTACTGCCTGGAGTACTTTCCTTCTCAGATGCTGCGTACCGGAAACAACTTT
ACCTTCAGCTACACTTTTGAGGACGTTCCTTTCCACAGCAGCTACGCTCACAGCCAGAGTCTGGA
CCGTCTCATGAATCCTCTCATCGACCAGTACCTGTATTACTTGAGCAAGACTATTAACGGTTCTGGA
CAGAATCAACAAACGCTAAAATTTTCTCAGGCCGGAGCGAGTGACATTCGGGACCAGTCTAGGAA
CTGGCTTCCTGGACCCTGTTACCGCCAGCAGCGAGTATCAACCACTGTGACTCAAAACAACAACA
GTGAATACTCGTGGACTGGAGCTACCAAGTACCACCTCAATGGCAGAGACTCTCTGGTGAATCCG
GGCCCGGCCATGGCAAGCCACAAGGACGATGAAGAAAAGTTTTTTCCTCAGAGCGGGGTTCTCAT
CTTTGGGAAGCAAGGCTCAGAGAAAACAAATGTGGACATTGAAAAGGTCATGATTACAGACGAA
GAGGAAATCAGGACAACCAATCCCGTGGCTACGGAGCAGTATGGTTCTGTATCTACCAACCTCCA
GGCTGGCAACAGACAAGCAGCTACCGCAGATGTCAACACACAAGGCGTTCTTCCAGGCATGGTC
TGGCAGGACAGAGATGTGTACCTTCAGGGGCCCATCTGGGCAAAGATTCCACACACGGACGGAC
ATTTTCACCCCTCTCCCCTCATGGGTGGATTCGGACTTAAACACCCTCCTCCACAGATTCTCATCAA
GAACACCCCGGTACCTGCGAATCCTTCGACCACCTTCAGTGCGGCAAAGTTTGCTTCCTTCATCAC
ACAGTACTCCACGGGACAGGTCAGCGTGGAGATCGAGTGGGAGCTGCAGAAGGAAAACAGCAA
ACGCTGGAATCCCGAAATTCAGTACACTTCCAACTACAACAAGTCTGTTAATGTGGACTTTACTGT
GGACACTAATGGCGTGTATTCAGAGCCTCGCCCCATTGGCACCAGATACCTGACTCGTAATCTGTA
A
(VP1 nucleic acid sequence of mutant 3 (5′->3′))
SEQ ID NO: 12
ATGGCTGCCGATGGTTATCTTCCAGATTGGCTCGAGGACACTCTCTCTGAAGGAATAAGAC
AGTGGTGGAAGCTCAAACCTGGCCCACCACCACCAAAGCCCGCAGAGCGGCATAAGGACGACAG
CAGGGGTCTTGTGCTTCCTGGGTACAAGTACCTCGGACCCTTCAACGGACTCGACAAGGGAGAGC
CGGTCAACGAGGCAGACGCCGCGGCCCTCGAGCACGACAAAGCCTACGACCGGCAGCTCGACA
GCGGAGACAACCCGTACCTCAAGTACAACCACGCCGACGCGGAGTTTCAGGAGCGCCTTAAAGA
AGATACGTCTTTTGGGGGCAACCTCGGACGAGCAGTCTTCCAGGCGAAAAAGAGGGTTCTTGAA
CCTCTGGGCCTGGTTGAGGAACCTGTTAAGACGGCTCCGGGAAAAAAGAGGCCGGTAGAGCACT
CTCCTGTGGAGCCAGACTCCTCCTCGGGAACCGGAAAGGCGGGCCAGCAGCCTGCAAGAAAAAG
ATTGAATTTTGGTCAGACTGGAGACGCAGACTCAGTACCTGACCCCCAGCCTCTCGGACAGCCAC
CAGCAGCCCCCTCTGGTCTGGGAACTAATACGATGGCTACAGGCAGTGGCGCACCAATGGCAGAC
AATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCT
ACAAACAAATTTCCAGCCAATCAGGAGCCTCGAACGACAATCACTACTTTGGCTACAGCACCCCT
TGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGA
GGTCACGCAGAATGACGGTACGACGACGATTGCCAATAACCTTACCAGCACGGTTCAGGTGTTTA
CTGACTCGGAGTACCAGCTCCCGTACGTCCTCGGCTCGGCGCATCAAGGATGCCTCCCGCCGTTCC
CAGCAGACGTCTTCATGGTGCCACAGTATGGATACCTCACCCTGAACAACGGGAGTCAGGCAGTA
GGACGCTCTTCATTTTACTGCCTGGAGTACTTTCCTTCTCAGATGCTGCGTACCGGAAACAACTTT
ACCTTCAGCTACACTTTTGAGGACGTTCCTTTCCACAGCAGCTACGCTCACAGCCAGAGTCTGGA
CCGTCTCATGAATCCTCTCATCGACCAGTACCTGTATTACTTGAGCAAGACTATTAACGGTTCTGGA
CAGAATCAACAAACGCTAAAATTTTCTCAGGCCGGAGCGAGTGACATTCGGGACCAGTCTAGGAA
CTGGCTTCCTGGACCCTGTTACCGCCAGCAGCGAGTATCAACCACTGTGACTCAAAACAACAACA
GTGAATACTCGTGGACTGGAGCTACCAAGTACCACCTCAATGGCAGAGACTCTCTGGTGAATCCG
GGCCCGGCCATGGCAAGCCACAAGGACGATGAAGAAAAGTTTTTTCCTCAGAGCGGGGTTCTCAT
CTTTGGGAAGCAAGGCTCAGAGAAAACAAATGTGGACATTGAAAAGGTCATGATTACAGACGAA
GAGGAAATCAGGACAACCAATCCCGTGGCTACGGAGCAGTATGGTTCTGTATCTACCAACCTCCA
GAGACAAGCAGCTACCGCAGATGTCAACACACAAGGCGTTCTTCCAGGCATGGTCTGGCAGGAC
AGAGATGTGTACCTTCAGGGGCCCATCTGGGCAAAGATTCCACACACGGACGGACATTTTCACCC
CTCTCCCCTCATGGGTGGATTCGGACTTAAACACCCTCCTCCACAGATTCTCATCAAGAACACCCC
GGTACCTGCGAATCCTTCGACCACCTTCAGTGCGGCAAAGTTTGCTTCCTTCATCACACAGTACTC
CACGGGACAGGTCAGCGTGGAGATCGAGTGGGAGCTGCAGAAGGAAAACAGCAAACGCTGGAA
TCCCGAAATTCAGTACACTTCCAACTACAACAAGTCTGTTAATGTGGACTTTACTGTGGACACTAA
TGGCGTGTATTCAGAGCCTCGCCCCATTGGCACCAGATACCTGACTCGTAATCTGTAA

1. A mutant of an adeno-associated virus 2 (AAV2) capsid protein, comprising:
an amino acid sequence KTINGSGQNQQTLK (SEQ ID NO: 2) or an amino acid sequence having 1, 2, 3, or 4 amino acid changes when compared to SEQ ID NO: 2 in variable region IV; and
an amino acid sequence TTVTQ (SEQ ID NO: 3) or an amino acid sequence having 1 or 2 amino acid changes when compared to SEQ ID NO: 3 in variable region V.

2. The mutant of the AAV2 capsid protein according to claim 1, wherein 12-16 consecutive amino acids in the variable region IV of a wild-type AAV2 capsid protein are replaced by the amino acid sequence KTINGSGQNQQTLK (SEQ ID NO: 2) or the amino acid sequence having 1, 2, 3, or 4 amino acid changes when compared to SEQ ID NO: 2, and
wherein 4-6 consecutive amino acids in the variable region V of the wild-type AAV2 capsid protein are replaced by the amino acid sequence TTVTQ (SEQ ID NO: 3) or an amino acid sequence having 1 or 2 amino acid changes when compared to SEQ ID NO: 3.

3. The mutant of the AAV2 capsid protein according to claim 1, wherein amino acids at positions 447-461 of a wild-type AAV2 capsid protein are replaced by the amino acid sequence KTINGSGQNQQTLK (SEQ ID NO: 2) or the amino acid sequence having 1, 2, 3, or 4 amino acid changes when compared to SEQ ID NO: 2,
wherein amino acids at positions 490-494 of the wild-type AAV2 capsid protein are replaced by the amino acid sequence TTVTQ (SEQ ID NO: 3) or the amino acid sequence having 1 or 2 amino acid changes when compared to SEQ ID NO: 3, and
wherein the positions of the amino acids correspond to positions in an amino acid sequence of the wild-type VP1 protein set forth in SEQ ID NO: 1.

4. The mutant of the AAV2 capsid protein according to claim 2, wherein amino acids at positions 447-461 of the wild-type AAV2 capsid protein are replaced by the amino acid sequence KTINGSGQNQQTLK (SEQ ID NO: 2) or the amino acid sequence having 1, 2, 3, or 4 amino acid changes when compared to SEQ ID NO: 2,
wherein amino acids at positions 490-494 of the wild-type AAV2 capsid protein are replaced by the amino acid sequence TTVTQ (SEQ ID NO: 3) or the amino acid sequence having 1 or 2 amino acid changes when compared to SEQ ID NO: 3, and
wherein the positions of the amino acids correspond to positions in an amino acid sequence of the wild-type VP1 protein set forth in SEQ ID NO: 1.

5. The mutant of the AAV2 capsid protein according to claim 1, wherein an amino acid sequence RTNTPSGTTTQSRLQ (SEQ ID NO: 4) in the variable region IV of a wild-type AAV2 capsid protein is replaced by the amino acid sequence KTINGSGQNQQTLK (SEQ ID NO: 2) or the amino acid sequence having 1, 2, 3, or 4 amino acid changes when compared to SEQ ID NO: 2, and
wherein an amino acid sequence KTSAD (SEQ ID NO: 5) in the variable region V of the wild-type AAV2 capsid protein is replaced by the amino acid sequence TTVTQ (SEQ ID NO: 3) or the amino acid sequence having 1 or 2 amino acid changes when compared to SEQ ID NO: 3.

6. The mutant of the AAV2 capsid protein according to claim 1, wherein the amino acid at position 585 is a non-basic amino acid, and wherein the position of the amino acid corresponds to a position in an amino acid sequence of the wild-type VP1 protein set forth in SEQ ID NO: 1.

7. The mutant of the AAV2 capsid protein according to claim 1, wherein arginine (R) at position 585 of a wild-type AAV2 capsid protein is mutated to alanine (A), and wherein the position of the arginine or alanine corresponds to a position in an amino acid sequence of the wild-type VP1 protein set forth in SEQ ID NO: 1.

8. The mutant of the AAV2 capsid protein according to claim 1, wherein amino acids at positions 585-587 of a wild-type AAV2 capsid protein are deleted, and wherein the positions of the amino acids correspond to positions in an amino acid sequence of the wild-type VP1 protein set forth in SEQ ID NO: 1.

9. The mutant of the AAV2 capsid protein according to claim 1, which is a mutant of capsid protein VP1, VP2 and/or VP3.

10. The mutant of the AAV2 capsid protein according to claim 1, comprising an amino acid sequence set forth in any one of SEQ ID NOs: 6-8, or an amino acid sequence having at least 80%, at least 90%, at least 95%, or at least 98% sequence identity to the amino acid sequence set forth in any one of SEQ ID NOs: 6-8.

11. An isolated nucleic acid molecule encoding the mutant of the AAV2 capsid protein according to claim 1.

12. The isolated nucleic acid molecule according to claim 11, comprising a nucleotide sequence set forth in any one of SEQ ID NOs: 10-12.

13. An expression vector comprising the isolated nucleic acid molecule according to claim 11.

14. An expression vector comprising the isolated nucleic acid molecule according to claim 12.

15. A host cell comprising the isolated nucleic acid molecule according to claim 11.

16. A host cell comprising the expression vector according to claim 13.

17. A host cell, which expresses the mutant of the AAV2 capsid protein according to claim 1.

18. An adeno-associated virus (AAV) comprising the mutant of the AAV2 capsid protein according to claim 1.

19. A method for preparing a recombinant adeno-associated virus (rAAV), comprising:
introducing at least the following components into a host cell:
(1) the isolated nucleic acid molecule according to claim 11 or an expression vector comprising the isolated nucleic acid molecule according to claim 11; and
(2) a GOI (gene of interest) plasmid comprising a target gene.

20. The method according to claim 19, wherein an expression product of the target gene is protein or RNA.

21. A rAAV prepared by the method according to claim 19.

22. The rAAV according to claim 21, which has lower targeting to liver than a wild-type AAV2 or wild-type AAV9.

23. The rAAV according to claim 21, which has higher targeting to muscle, heart, brain, spinal cord, lung, kidney, or eye than a wild-type AAV2 or wild-type AAV9.

24. A pharmaceutical composition comprising the rAAV according to claim 21 and a pharmaceutically acceptable carrier.

25. A pharmaceutical composition comprising the isolated nucleic acid molecule according to claim 11 or an expression vector comprising the isolated nucleic acid molecule according to claim 11 and a pharmaceutically acceptable carrier.

26. A method for treating a muscle, heart, brain, spinal cord, lung, kidney, or eye-related disease, comprising:
administering a therapeutically effective amount of the pharmaceutical composition according to claim 25 to a patient suffering from the muscle, heart, brain, spinal cord, lung, kidney, or eye-related disease, wherein the pharmaceutical composition comprises a heterologous polynucleotide encoding a heterologous gene product.

27. A method for treating a muscle, heart, brain, spinal cord, lung, kidney, or eye-related disease, comprising:
administering a therapeutically effective amount of the pharmaceutical composition according to claim 24 to a patient suffering from the muscle, heart, brain, spinal cord, lung, kidney, or eye-related disease, wherein the pharmaceutical composition comprises a heterologous polynucleotide encoding a heterologous gene product.


SEQUENCE LISTING (26 sequences)

SEQ ID NO: 1 - protein, 735 aa, Adeno-associated dependoparvovirus A [sequence omitted]

SEQ ID NO: 2 - protein, 14 aa, synthetic construct
KTINGSGQNQQTLK

SEQ ID NO: 3 - protein, 5 aa, synthetic construct
TTVTQ

SEQ ID NO: 4 - protein, 15 aa, synthetic construct
RTNTPSGTTTQSRLQ

SEQ ID NO: 5 - protein, 5 aa, synthetic construct
KTSAD

SEQ ID NO: 6 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSTTVTQNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSTTVTQNNNSEYSWTGATKYHLNGRDSLVNPGPAMASHKDDEEKFFPQSGVLIFGKQGSEKTNVDIEKVMITDEEEIRTTNPVATEQYGSVSTNLQAGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 731 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSTTVTQNNNSEYSWTGATKYHLNGRDSLVNPGPAMASHKDDEEKFFPQSGVLIFGKQGSEKTNVDIEKVMITDEEEIRTTNPVATEQYGSVSTNLQRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 2208 bp, Adeno-associated dependoparvovirus A
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 10 - DNA, 2205 bp, synthetic construct
  source 1..2205: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaccactgtgactcaaaacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 11 - DNA, 2205 bp, synthetic construct
  source 1..2205: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaccactgtgactcaaaacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggctggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 12 - DNA, 2196 bp, synthetic construct
  source 1..2196: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaccactgtgactcaaaacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 13 - DNA, 35 bp, synthetic construct
  source 1..35: mol_type = other DNA
catctttgaacaataaatgatttaaatcaggtatg

SEQ ID NO: 14 - DNA, 25 bp, synthetic construct
  source 1..25: mol_type = other DNA
tcaactgaaacgaatcaaccggttt

SEQ ID NO: 15 - DNA, 59 bp, synthetic construct
  source 1..59: mol_type = other DNA
tgttgattctgtccagaaccgttaatagtcttgctcaagtaatacaggtactggtcgat

SEQ ID NO: 16 - DNA, 53 bp, synthetic construct
  source 1..53: mol_type = other DNA
acggttctggacagaatcaacaaacgctaaaattttctcaggccggagcgagt

SEQ ID NO: 17 - DNA, 46 bp, synthetic construct
  source 1..46: mol_type = other DNA
gttgttttgagtcacagtggttgatactcgctgctggcggtaacag

SEQ ID NO: 18 - DNA, 50 bp, synthetic construct
  source 1..50: mol_type = other DNA
atcaaccactgtgactcaaaacaacaacagtgaatactcgtggactggag

SEQ ID NO: 19 - DNA, 44 bp, synthetic construct
  source 1..44: mol_type = other DNA
ttgtctgttgccagcctggaggttggtagatacagaaccatact

SEQ ID NO: 20 - DNA, 37 bp, synthetic construct
  source 1..37: mol_type = other DNA
cctccaggctggcaacagacaagcagctaccgcagat

SEQ ID NO: 21 - DNA, 39 bp, synthetic construct
  source 1..39: mol_type = other DNA
ctgcttgtctctggaggttggtagatacagaaccatact

SEQ ID NO: 22 - DNA, 31 bp, synthetic construct
  source 1..31: mol_type = other DNA
caacctccagagacaagcagctaccgcagat

SEQ ID NO: 23 - DNA, 20 bp, synthetic construct
  source 1..20: mol_type = other DNA
aaccagcgccattctgatca

SEQ ID NO: 24 - DNA, 20 bp, synthetic construct
  source 1..20: mol_type = other DNA
tcggggttgttaacgtagcc

SEQ ID NO: 25 - DNA, 21 bp, synthetic construct
  source 1..21: mol_type = other DNA
caggagagtgtttcctcgtcc

SEQ ID NO: 26 - DNA, 20 bp, synthetic construct
  source 1..20: mol_type = other DNA
ttcccattctcggccttgac